(19) United States
(12) Patent Application Publication
(10) Pub. No.: US 2010/0174137 A1
(43) Pub. Date: Jul. 8, 2010

(54) Adaptive stimulation systems and methods

(76) Inventor: Youngtack Shim, Port Moody (CA)

Correspondence Address: Youngtack Shim, 155 Aspenwood Drive, Port Moody, V3H 5A5, CA

(21) Appl. No.: 12/318,668

(22) Filed: Jan. 6, 2009

(51) Int. Cl. A61F 5/00; A61N 1/36

(52) U.S. Cl. 600/38; 607/39

(57) ABSTRACT

The present invention generally relates to systems capable of providing and delivering electric potential to a pelvic (i.e., vaginal) structure of a female to cause contraction of pelvic muscles and/or stimulation of pelvic nerves. More particularly, the present invention relates to various systems which include various power members and electrode members, where such power members are capable of providing electric power for such potential from various sources and where such electrode members are capable of delivering the potential along a preset direction or multiple directions. Therefore, such systems may also induce contraction of the pelvic muscles and/or stimulation of the pelvic nerves in a desirable order. Such systems may further induce the muscle contraction and/or nerve stimulation in synchronization with dynamic patterns of various variables of the system. The present invention also relates to various methods of generating and delivering such potential to the pelvic structure, causing the muscle contraction and/or nerve stimulation in a desirable order, and synchronizing such muscle contraction and/or nerve stimulation with movement of such systems. The present invention further relates to various processes for providing various members, units, and/or parts of such systems.

### CROSS-REFERENCES TO RELATED APPLICATIONS

The present application claims an earlier invention date of the Disclosure Document entitled the same, deposited in the U.S. Patent and Trademark Office (the “Office”) on Jan. 23, 2007 under the Disclosure Document Deposit Program (“DDDP”) of the Office, and bearing the Ser. No. 611,334. The present application also claims earlier invention dates of other Disclosure Documents, the first of which is entitled “Dynamic control relaxing systems and methods,” deposited in the Office on Jan. 12, 2007 under the DDDP, and bearing the Ser. No. 611,023, the second of which is entitled “Audio relaxing systems and methods, deposited in the Office on Jan. 23, 2007 under the DDDP, and bearing the Ser. No. 611,331, the third of which is entitled “Synchronized relaxing systems and methods,” deposited in the Office on Jan. 12, 2007 under the DDDP, and bearing the Ser. No. 611,027, and the fourth of which is entitled “Pelvic exercise systems and methods,” deposited in the Office on Jan. 12, 2007 under the DDDP, and bearing the Ser. No. 611,016. It is to be appreciated that an entire portion of each of the above Disclosure Documents is incorporated herein by reference.

### CROSS-REFERENCES TO RELATED APPLICATIONS

The present application claims an earlier invention date of the Disclosure Document entitled the same, deposited in the U.S. Patent and Trademark Office (the “Office”) on Jan. 12, 2007 under the Disclosure Document Deposit Program of the Office, and bearing the Ser. No. 611,025 an entire portion of which is incorporated herein by reference.

### FIELD OF THE INVENTION

The present invention generally relates to systems capable of providing and delivering electric potential to a pelvic (i.e., vaginal) structure of a female to cause contraction of pelvic muscles and/or stimulation of pelvic nerves. More particularly, the present invention relates to various systems which include various power members and electrode members, where such power members are capable of providing electric power for such potential from various sources and where such electrode members are capable of delivering the potential along a preset direction or multiple directions. Therefore, such systems may also induce contraction of the pelvic muscles and/or stimulation of the pelvic nerves in a desirable order. Such systems may further induce the muscle contraction and/or nerve stimulation in synchronization with dynamic patterns of various variables of the system. The present invention also relates to various methods of generating and delivering such potential to the pelvic structure, causing the muscle contraction and/or nerve stimulation in a desirable order, and synchronizing such muscle contraction and/or nerve stimulation with movement of such systems. The present invention further relates to various processes for providing various members, units, and/or parts of such systems.

### BACKGROUND OF THE INVENTION

A vaginal anatomy typically includes a vaginal entry and a vaginal wall, where such an entry defines an orifice therethrough, while the wall includes muscles and defines a vaginal cavity which extends inwardly from the entry and which is also bound by such muscles. The entry forms a clitoris thereon, and a paraurethral gland of an urethral sponge of a clitoris (also called the Grafenberg spot or G spot) is believed to be defined on the wall. The vaginal wall is formed essentially of two sets of muscles, the former extending longitudinally while the latter encircling the vagina. These muscles are specifically termed as “pubococygenus” and “levator ani” and are located immediately adjacent to the vagina. These muscles have general appearance of a hammock with its two ends connected to the sides of a pelvis. In particular, the pubococygenus is basically a sphincter muscle which passes through a middle third of the vagina and runs in a circular band, with a ring-like ridges forming a part of a urethra and anus. For simplicity of illustration, this vaginal anatomy is to be referred to as “a pelvic structure,” the vaginal entry as an “entry,” and the vaginal wall as a “wall.” In addition, such a pelvic structure is referred to as the “standard pelvic structure” or simply the “pelvic structure” hereinafter for simplicity of illustration, unless otherwise specified.

Among devices currently available in the market for enhancing sexual functioning are dildos, vaginal exercise bars, and prostate stimulators. These devices generally provide stimuli from friction upon manipulation of their stimulators or by pressure due to distention of the pelvic cavity effected by a volume of such stimulators.

For example, manual pelvic relaxing devices have been proposed in various configurations as disclosed in various prior art such as, e.g., U.S. Pat. No. 3,996,930 to Sekulich, U.S. Pat. No. 5,690,603 to Kain, U.S. Pat. No. 5,690,604 to Barnett, U.S. Pat. No. 5,853,362 to Jacobs, U.S. Pat. No. 6,203,491 to Uribe, U.S. Pat. App. Pub. No. 2005/0187431 by Hudson, U.S. Pat. App. Pub. No. 2005/0228218 by Skidmore et al., U.S. Pat. No. 6,540,667 to Hickman, and the like. Being manual, users have to manually move such devices in and out of the pelvic cavity and/or around the pelvic opening.

In order to overcome inconvenience thereof, various automatic mechanisms have been added to such pelvic relaxing devices. In one class of examples, electric motors or electromagnetic vibration mechanisms have been incorporated to various automatic devices for effecting vibration as disclosed in various prior art such as, e.g., U.S. Pat. No. 3,451,391 to Tavel, U.S. Pat. No. 3,504,665 to Bakunin et al., U.S. Pat. No. 3,626,931 to Bysakh, U.S. Pat. No. 3,669,100 to Csanad, U.S. Pat. No. 3,991,751 to O'Rourke, U.S. Pat. No. 4,788,968 to Rudashevsky et al., U.S. Pat. No. 5,067,480 to Woog et al, U.S. Pat. No. 6,056,705 to Stigar-Brown, U.S. Pat. Appl. Pub. 2004/0034315 to Chen, U.S. Pat. Appl. Pub. 2004/0127766 to Chen, and the like.

In another class of example, automatic pelvic relaxing devices have used various mechanisms of converting rotational movements generated by such electric motors into translational movements for effecting horizontal and/or vertical translation of their stimulators. Several examples of such prior art include U.S. Pat. No. 4,722,327 to Harvey, U.S. Pat. No. 4,790,296 to Segal, U.S. Pat. No. 5,076,261 to Black, U.S. Pat. No. 5,725,473 to Taylor, U.S. Pat. No. 6,142,929 to Padgett, U.S. Pat. No. 6,422,993 to Hudson, U.S. Pat. No. 6,866,645 to Lee, and U.S. Pat. Appl. Pub. 2004/0147858

Various automatic pelvic relaxing devices have also used various mechanisms for converting rotational movements generated by such electric motors into lateral movements for effecting horizontal translation of their stimulators along a direction generally normal to axes of such stimulators. Several examples of such prior art are U.S. Pat. No. 5,460,597 to Hopper, U.S. Pat. No. 5,470,303 to Leonard et al., and U.S. Pat. No. 5,851,175 to Nickell.

Other therapeutic devices, although developed for various purposes other than pelvic relaxing, seem to have been used as alternatives as such conventional pelvic relaxing devices. In one class of examples, various manual or vibration devices have been disclosed to train or heal pelvic muscles as exemplified in U.S. Pat. No. 3,598,106 to Buning, U.S. Pat. No. 4,241,912 to Mercer et al., and U.S. Pat. No. 4,574,791 to Mitchener. In another class of examples, various devices have been developed for massaging various portions of a human body as disclosed in U.S. Pat. No. 4,055,170 to Nohmura, U.S. Pat. No. 4,825,853 to Iwamoto et al., U.S. Pat. No. 4,846,158 to Teranishi, U.S. Pat. No. 4,911,149 to Borodulin et al., U.S. Pat. No. 5,063,911 to Teranishi, and the like. In another class, massage devices have also been devised to provide translational movements as disclosed in U.S. Pat. No. 4,002,164 to Bradley, U.S. Pat. No. 5,085,207 to Fiore, U.S. Pat. No. 5,676,637 to Lee, and the like. In another class of examples, various devices have also been arranged to provide rotating, tapping, swinging and/or swiveling movements as described in U.S. Pat. No. 4,162,675 to Kawada, U.S. Pat. No. 6,632,185 to Chen, U.S. Pat. No. 4,088,128 to Mabuchi, U.S. Pat. No. 4,513,737 to Mabuchi, U.S. Pat. No. 4,827,914 to Kanazawa, U.S. Pat. No. 4,834,075 to Guo et al., U.S. Pat. No. 5,183,034 to Yamasaki et al., and U.S. Pat. No. 6,402,710 to Hsu. A vacuum device of U.S. Pat. No. 4,033,338 to lgwebike as well as a balloon device of U.S. Pat. No. 4,050,449 to Castellana et al. have also been proposed.

Regardless of their detailed mechanisms and/or movements effected thereby, all of these prior art devices suffer from common drawbacks. Excluding those manual ones, typical automatic devices consist of main modules and control modules which operatively couple with the main modules by wire for delivering electric power and control signals. Such wire, however, tends to be easily tangled and damaged. To overcome this defect, modern automatic pelvic relaxing device are fabricated as single unitary articles each with a main body and a handle which fixedly couples with a top part of the main body. The main body is generally designed to be inserted into the pelvic cavity, whereas the handle is shaped and sized to provide a grip for the user and also incorporates therein various control buttons. Accordingly, the handle consists of a space to form the grip and another space for such buttons. In order to avoid providing an inadvertently long device, however, a part of the handle closer to the main body is recruited to define the grip, whereas the rest of the handle houses the control buttons. It is to be appreciated, however, that all control buttons of conventional automatic devices are either on/off switches or speed control switches, where the on/off switches turn on and off the entire device or a specific movement thereof, and the speed control switches control a speed of the specific movement. In addition, such switches are typically designed to be activated and deactivated each time the user presses or touches them. Accordingly, when the user inadvertently touches any of such on/off and control switches during use, the device may be accidentally turned off, change speeds, and the like. In order to avoid such inadvertent operation, the control buttons have been incorporated as far away from the grip space of the handle, which in turn causes the very inconvenience of requiring the user to change the grip or to move his or her hand to manipulate the control buttons during operation when the user wants to change the speed of movement.

In contrary to these devices, novel pelvic relaxing systems, methods, and/or processes have already been conceived of and disclosed in numerous co-pending Applications of the same Applicant. For example, various pelvic relaxing systems have been proposed for manipulating their input and/or sensor units without mandating the user to change the grip, for providing various stimuli to the clitoris and/or G-spot of the user, for providing interactive capabilities thereto, for synchronizing movements of their various parts and/or operations thereof with internal and/or external signals, for incorporating electric stimulators thereinto, for installing the body members capable of adjusting their configurations, for incorporating retention mechanisms thereinto, for providing feedback mechanisms thereto, and for generating reciprocating movements of only portions of body members thereof. Although these novel systems solve most deficiencies of the conventional devices, none of them are capable of assisting the user in improving muscle tones of her pelvic structure.

It is well recognized in the field that improved muscle strengths and tones of various muscles of the pelvic structure are advantageous in many respects. For example, the stronger pelvic muscles are generally attributed to experiencing an orgasm or to having more intense and/or multiple orgasms during a sexual intercourse. To this end, various modalities have been proposed to improve tones of the pelvic muscles among which the most popular is the “Kegel” exercise. Similar to other exercising modalities, the Kegel exercise hinges on a well-accepted principle of “Specific Adaptation to Imposed Demand” which dictates that muscles will adapt only to demands placed thereupon. Accordingly, one need to place an ever-increasing demand on her muscles for continued improvement thereof, e.g., by adding an extra repetition to an exercise, increasing demand (or load) on the muscles, shortening rest intervals during the exercise, and the like. Accordingly, various conventional pelvic exercise devices urge the user to add progressive dynamic resistance to conventional Kegel exercise devices.

Although conventional Kegel exercise devices are generally effective in improving the muscle strengths and tones, such devices suffer from several common deficiencies. For example, almost all conventional devices use sensors filled with air, and assess physiologic states of various muscles by monitoring changes in pressure of air trapped inside the sensors. The sensor is generally disposed in an insertable unit of the device and arranged to be in fluid communication with a monitoring unit such as an analog pressure gauge or a digital display through a tubing which is typically made of polymers. Because air is a compressible gas, however, such air pressure inside the sensor generally depends upon temperature of a surrounding medium. Therefore, the air trapped in the sensor is heated to body temperature upon insertion of the insertable unit into the internal cavity of the structure, and expands due to an increase in temperature. Because the tubing is disposed in room temperature, however, a mismatch between such temperatures may cause measurement error in the air pressure. In addition, the compliant tubing between the sensor and monitoring unit may change its internal volume when the tubing is moved around. In addition, such a conventional device is typically forced to employ a single sensor, for it is not readily amenable to incorporate two compliant air-filled sensors. Therefore, such a device employs the sensor over at least a substantial part of its insertable unit and only provides a value of the air pressure averaged over the part of the sensor. Moreover, such conventional devices are directed to measure the pressure exerted by the pelvic muscles while the user maintains a fixed posture and cannot assess configurational changes in the pelvic structure as the user is in a different posture.

Although all prior art devices proclaim to enhance pleasure in pelvic relaxing activities, most of them are geared toward providing mechanical stimulation, while only a handful of such employ electric stimulation mechanisms. However, the latter devices generally deliver electric stimuli in fixed patterns such as, e.g., a fixed number of electric stimuli per minute. In addition, the electric stimulation devices may only produce identical stimuli over time, with the only possible option of changing their amplitudes. Moreover, such devices generate the electric stimuli by electrical power supplied from their batteries. It is, therefore, manifest that none of such electrical stimulation devices of the prior art may be able to deliver the electrical stimuli which may be in synchronization with various movements associated with pelvic relaxing activities such as masturbation and sexual intercourse, just to name a few. In addition, none of such electrical stimulation devices of the prior art may afford to be incorporated onto portable devices which may be worn around a penile structure of a male.

Accordingly, there is an impending need for an adaptive pelvic stimulation system capable of being worn around a penile structure of a male and delivering electric potential to a pelvic structure of a female. There also is an urgent need for an adaptive pelvic stimulation system capable of delivering such electric potential in synchronization with various sexual activities. There further is an impending need for an adaptive pelvic stimulation system capable of generating power for such potential from a variety of sources all of which may preferably facilitate implementation of such a system onto a penile structure of a male.

### SUMMARY OF THE INVENTION

The present invention generally relates to systems capable of providing and delivering electric potential to a pelvic (i.e., vaginal) structure of a female to cause contraction of pelvic muscles and/or stimulation of pelvic nerves. More particularly, the present invention relates to various systems which include various power members and electrode members, where such power members are capable of providing electric power for such potential from various sources and where such electrode members are capable of delivering the potential along a preset direction or multiple directions. Therefore, such systems may also induce contraction of the pelvic muscles and/or stimulation of the pelvic nerves in a desirable order. Such systems may further induce the muscle contraction and/or nerve stimulation in synchronization with dynamic patterns of various variables of the system and/or the pelvic structure, where various sensor units of the systems may monitor movements of various members and/or units of the systems, dispositions thereof, and/or external perturbations and where control members of the systems may manipulate a timing of such potential, an amplitude thereof, and other dynamic features of such potential. Such systems may preferably obtain the power from various portable and compact sources such as, e.g., portable electromechanical generator capable of converting movement of the system and/or contact force applied thereto into electrical energy, portable piezoelectric generator for converting the contact force applied thereto into electrical energy, portable and mobile magnet and/or electric conductor moving across electric and/or magnetic fields and generating the potential through electromagnetic induction.

The present invention also relates to various methods of generating such electric potential and delivering such potential to the pelvic structure for inducing contraction of muscles and/or stimulation of nerves of the pelvic structure. More particularly, the present invention relates to various methods of providing various portable adaptive stimulation systems, generating power for such potential from various portable sources, delivering such potential by various electrode members, arranging multiple electrode members, and the like. The present invention also relates to various methods of measuring various system and/or structure variables and delivering such potential when values of the variables exceed a preset value (or fall within a preset range) and various methods of generating or delivering such potential to the pelvic structure in synchronization with such system and/or structure variables. The present invention also relates to various methods of synchronizing the muscle contraction and/or nerve stimulation with one or more variables of the system and/or structure, and various methods of inducing the muscle contraction and/or nerve stimulation in a preset order and/or pattern. The present invention further relates to various processes for providing various members, units, and/or parts of the system and, more particularly, various processes for making portable power members capable of supplying power for the potential, for incorporating the power members into wearable body members of the system, for incorporating various electrode members onto such body members, for providing control members capable of measuring various system and/or structure variables and synchronizing such potential with such variables, and the like.

Therefore, one objective of the present invention is to provide an adaptive stimulation system which may contract muscles of the pelvic structure and/or stimulate nerves of such a structure with electric potential. Thus, a related objective of the present invention is to provide such a system which may be releasably coupled to and/or disposed over at least a portion of a pelvic structure of a female. Another related objective of the present invention is to form such a system which may be releasably coupled to and/or disposed over or around at least a portion of a penile structure of a male. Another related objective of this invention is to provide the system at least one part of which may be similarly coupled and/or disposed in the pelvic structure, while at least another part of which may be similarly coupled and/or disposed in the penile structure.

Another objective of the present invention is to fabricate another adaptive stimulation system which may be releasably coupled to and/or disposed around at least a portion of a pelvic structure of a female and/or at least a portion of a penile structure of a male, and deliver electric potential to such a portion of the pelvic structure. Thus, a related objective of the present invention is to provide such a system capable of being disposed over and/or worn along the penile structure of the male similar to a conventional condom while delivering such potential. Another related objective of this invention is to provide such a system which may be disposed into and/or worn around such a pelvic structure while delivering such potential.

Another objective of the present invention is to provide an adaptive stimulation system which may be releasably coupled to and/or disposed around at least a portion of one or both of such pelvic and/or penile structures, generate electric potential, and then deliver such potential to such a portion of the pelvic structure. Thus, a related objective of the present invention is to provide such a system capable of being disposed over and/or worn along the penile structure while generating and delivering such potential. Another related objective of this invention is to provide such a system which may be similarly disposed into and/or worn around such a pelvic structure while generating and delivering the potential. Another related objective of this invention is to provide a disposable system at least most parts of which may be discarded after use. Accordingly, another related objective of this invention is to provide such a system which is made of cheap materials by simple manufacturing processes or, in the alternative, to provide such a system at least a part of which may be reusable, while the rest of which may be discarded after use.

Another objective of the present invention is to provide an adaptive stimulation system which may be similarly coupled and/or disposed in the pelvic and/or penile structures, and provide power for generating such potential. Thus, a related objective of this invention is to fabricate a portable power member capable of converting movement of at least a part of the system into electric power and then generating such potential therefrom. Another related objective of this invention is to provide a portable power member capable of converting external force applied thereto into electric power and providing such potential therefrom. Another related objective of this invention is to fabricate a portable power member capable of generating electric power through electromagnetic induction and providing such potential therefrom. Another related objective of the present invention is to provide a system which may include a battery and draw a certain amount of electric energy stored therein for generating such potential.

Another objective of the present invention is to provide an adaptive stimulation system which may be also coupled and/or disposed in the pelvic and/or penile structures, and generate the potential by electromagnetic induction. Therefore, a related objective of the present invention is to provide the system including at least one field unit capable of generating the electric and/or magnetic fields and at least one movable unit which may be a magnet and/or an electric conductor, where the movable is arranged to move across such fields. Another related objective of this invention is to provide another system which defines such units and is disposed in a body member to be coupled and/or disposed in the penile structure. Another related objective of this invention is to provide a system which includes such units and is disposed in a body member to be coupled and/or disposed in the pelvic structure. Another related objective of this invention is to provide a system which defines at least two modules, and includes each of such units in such modules, where one of such modules is to be coupled and/or disposed in the penile structure, while the other of such modules is to be coupled and/or disposed in the pelvic structure. Another related objective of this invention is to provide a system which includes the movable unit which in turn is to move across external electric and/or magnetic fields.

Another objective of the present invention is to provide an adaptive stimulation system which may be similarly coupled and/or disposed in the pelvic and/or penile structures, generate the potential, and deliver such potential to such a portion of the pelvic structure based upon movement of at least a part of the system and/or structure, contact between such a part of the system with the structure or between multiple parts of the system, and/or disposition of such a part of the system. Thus, a related objective of the present invention is to provide the system capable of generating and/or delivering the potential when its sensor unit detects such movement. Another related objective of this invention is to provide a system capable of generating and/or delivering the potential when its sensor unit detects an external force and/or energy applied thereto. Another related objective of this invention is to provide a system capable of generating and/or delivering such potential when its sensor unit detects disposition of such a part of the system beyond a preset landmark or within a preset range.

Another objective of the present invention is to provide an adaptive stimulation system which may be similarly coupled and/or disposed in the pelvic and/or penile structures, generate the potential, and deliver the potential to the portion of the pelvic structure as measured values of such movement, force, and/or disposition exceed preset values and/or fall within preset ranges. Therefore, a related objective of this invention is to provide the system capable of generating and/or delivering the potential when a displacement of at least a part of such a system, velocity thereof, acceleration thereof, and/or other related dynamic patterns thereof may exceed the preset value or fall within the preset range. Another related objective of this invention is to provide a system capable of generating or delivering such potential when a momentum of such a part of the system and/or energy thereof may exceed the preset value or fall within the preset range. Another related objective of this invention is to provide a system capable of generating and/or delivering such potential when a magnitude and/or a duration of the contact force exceeds the preset value or falls within the preset range. Another related objective of this invention is to provide a system capable of generating or delivering the potential when such a part of the system is disposed beyond a preset landmark, within a preset range, and the like.

Another objective of the present invention is to provide an adaptive stimulation system which may be similarly coupled and/or disposed in the pelvic and/or penile structures, generate the potential, and deliver the potential to the portion of the pelvic structure in synchronization with such movement, force, and/or disposition. Thus, a related objective of the present invention is to provide the system capable of generating and/or delivering the potential in synchronization with various dynamic patterns of the movement of at least one part of the system and/or at least a portion of the structure. Another related objective of this invention is to provide a system capable of generating and/or delivering such potential in synchronization with various dynamic patterns of the external force and/or energy applied to such a part of the system and/or such a portion of the structure. Another related objective of this invention is to provide a system capable of generating or delivering such potential in synchronization with various dynamic patterns of such disposition of such a part of the system.

Another objective of the present invention is to provide an adaptive stimulation system which may be similarly coupled and/or disposed in the pelvic and/or penile structures, and then contract the muscles of the pelvic structure and/or stimulate the nerves thereof in preset directions. Therefore, a related objective of the present invention is to provide the system capable of providing the potential in one or more preset directions. Another related objective of this invention is to provide such a system capable of inducing such contraction and/or stimulation along an uniform direction(s). Another related objective of this invention is to provide a system capable of inducing the contraction and/or stimulation along alternating or opposite directions. Another related objective of this invention is to provide such a system capable of inducing such contraction and/or stimulation in a direction(s) determined by various system and/or structure variables, including movement of at least a part of the system, displacement thereof, velocity thereof, acceleration thereof, disposition thereof, orientation of the system, contact force applied thereto, and the like.

Another objective of the present invention is to provide an adaptive stimulation system which may be similarly coupled and/or disposed in the pelvic and/or penile structures, generate the potential, and deliver such potential while manipulating various dynamic features of such potential. Therefore, a related objective of the present invention is to provide a system capable of various dynamic features of such potential based on movement of at least a part of the system and/or at least a portion of such a pelvic structure. Another related objective of this invention is to provide another system capable of manipulating various dynamic features of the potential based on the external force applied onto such a part and/or portion. Another related objective of this invention is to provide another system capable of manipulating various dynamic features of such potential based on the disposition of such a part of the system.

Various aspects and/or embodiments of various systems, methods, and/or processes of this invention will now be described, where such aspects and/or embodiments only represent different forms. Such systems, methods, and/or processes of this invention, however, may also be embodied in many other different forms and, therefore, should not be limited to the aspects and/or embodiments which are set forth herein. Rather, various exemplary aspects and/or embodiments described herein are provided so that this disclosure will be thorough and complete, and fully convey the scope of the present invention to one of ordinary skill in the art. It is to be understood that various movements and mechanisms therefor as well as various control algorithms of the prior art devices as described in the above Background of the Invention are to be incorporated herein in their entireties by reference.

In one aspect of the present invention, such a stimulation system may be arranged to provide electric potential and to deliver the potential to at least one of muscles and nerves of a pelvic structure of a female which may include an entry and a wall, where the entry forms therethrough an orifice and where the wall is arranged to include the muscles and nerves and to form an internal cavity extending inwardly and bound by the muscles. Such a pelvic structure is to be referred to as a “standard pelvic structure” or the “pelvic structure” throughout this description, unless otherwise specified.

In one exemplary embodiment of this aspect of the present invention, a system may include at least one first body member, at least one power member, and at least one electrode member. Such a first body member may be arranged to receive at least a portion of a penile structure of a male therein, where such a first body member is to be referred to as a “body member of the first type” hereinafter, unless otherwise specified. The power member may be arranged to provide electric power for such an potential, where such a power member is to be referred to as a “power member of the first type” hereinafter, unless otherwise specified. The electrode member may be disposed onto the first body member, to receive the potential from the power member, and then to deliver the potential to the pelvic structure therethrough when the first body member engages the pelvic structure, thereby resulting in contraction of the muscles of the pelvic structure and/or stimulation of the nerves of such a structure. Such an electrode member is to be referred to as a “electrode member of the first type” hereinafter, unless otherwise specified.

In another exemplary embodiment of this aspect of the present invention, a system may include at least one second body member, the power member of such a first type, and at least one electrode member. The second body member may be arranged to receive therein at least a portion of the pelvic structure, where such a second body member is to be referred to as a “body member of the second type” hereinafter, unless otherwise specified. Such an electrode member may be incorporated onto the second body member and may be arranged to receive the potential from the power member and to deliver such potential to the pelvic structure therethrough when the second body member engages a penile structure of a male, thereby causing contraction of the muscles of the pelvic structure and/or stimulation of the nerves of the same structure. Such an electrode member is to be referred to as an “electrode member of the second type” hereinafter, unless otherwise specified.

In another exemplary embodiment of this aspect of the present invention, such a system may have the body member of the first type, another body member of the second type, at least one power member, and at least one electrode member. The power member may be arranged to provide power for the potential and to be disposed in the first and/or second body members. The electrode member may be arranged to be disposed onto the first and/or second body members, to receive the potential from the power member, and to deliver the potential to the pelvic structure therethrough when such body members engage each other, thereby causing contraction of the muscles of the pelvic structure and/or stimulation of the nerves thereof.

In another exemplary embodiment of this aspect of the present invention, a system may include at least one first body member, the power member of the first type, and the electrode member of the first type, where such a first body member may be arranged to be worn onto an object which may be inserted into the internal cavity and/or disposed on the orifice.

In another exemplary embodiment of this aspect of the present invention, a system may include at least one first body member, the power member of the first type, and the electrode member of the first type, where the first body member may be arranged to be insertable into the internal cavity and/or disposed on the orifice.

In another aspect of the present invention, a stimulation system may have multiple modules and arranged to provide electric potential and to deliver the potential to at least one of muscles and nerves of the standard pelvic structure of a female.

In one exemplary embodiment of this aspect of the present invention, a system may include the first body member of the first type, the power member of the first type, at least one electrode member, a first module, and a second module. The electrode member may be arranged to be incorporated onto at least one of the first body member and power member, and to receive the potential from the power member. The first module may be arranged to include at least one of the members, while the second module may be arranged to include the rest of such members and to be releasably coupled to the first module. The electrode member may also be arranged to deliver such potential to the pelvic structure therethrough as the first and second modules operatively couple with each other and as the first body member and at least one of such modules including the first body therein engage the pelvic structure, thereby causing contraction of the muscles of the pelvic structure and/or stimulation of the nerves of such a structure.

In another exemplary embodiment of this aspect of the present invention, such a system may have the body member of the second type, the power member of the first type, at least one electrode member. Such an electrode member may be disposed in at least one of the second body member and power member and arranged to receive such potential from the power member. The first module may include at least one of the members, whereas the second module may include the rest of the members and arranged to releasably couple with the first module. Such an electrode member may be arranged to deliver the potential to the pelvic structure therethrough when the first and second modules may be operatively coupled to each other and as the first body member and at least one of the modules with the first body engage a penile structure of a male, thereby causing contraction of the muscles and/or stimulation of the nerves.

In another exemplary embodiment of this aspect of the present invention, a system may include the body member of the first type, the body member of the second type, the power member of the first type, a first module, and a second module. The first module may include the first body member, while the second module may include the second body member. The rest of the members may be arranged to be included in at least one of the body members, and where the electrode member may be arranged to deliver the potential to the pelvic structure therethrough when the first and second modules may be operatively coupled to each other and when the first body member and at least one of such modules having the first body therein engage the penile structure, thereby causing contraction of the muscles and/or stimulation of the nerves.

In another aspect of the present invention, another stimulation system may also be arranged to provide electric potential and to deliver the potential to muscles and/or nerves of the standard pelvic structure of a female in cooperation with a device which may be external to the system.

In one exemplary embodiment of this aspect of the present invention, a system may include at least one body member which may be a first body member which may be capable of receiving therein at least a portion of the pelvic structure and/or a second body member capable of receiving at least a portion of a penile structure of a male thereinto. Such a body member is to be referred to as a “body member of the third type” hereinafter, unless otherwise specified. The system may also include the power member of the first type, at least one electrode member, and at least one control member. The electrode member may be disposed on the body member of the third type and arranged to receive the potential from the power member. Such an electrode member will also be referred to as an “electrode member” of the third type hereinafter, unless otherwise specified. In addition, the control member may be arranged to obtain at least one external signal from the external device through wire or wirelessly, to operatively couple with the electrode and power members, and to deliver such potential through the electrode member to the pelvic structure based on the external signal as the body member of the third type engage at least one of the structures, thereby causing contraction of such muscles of the pelvic structure and/or stimulation of the nerves thereof.

In another exemplary embodiment of this aspect of the present invention, a system may include the body member of the third type, at least one electrode member, at least one control member, and so on. The electrode member may be disposed on the body member of the third type, while the control member may be arranged to receive power for the potential from the external device through wire or wirelessly, to operatively couple with the electrode member, and to deliver such potential through the electrode member to the pelvic structure as the body member of the third type engage at least one of the structures, thereby causing contraction of the muscles of the pelvic structure and/or stimulation of the nerves thereof.

In another aspect of the present invention, a stimulation system may be provided to be worn by a male, to provide electric potential, and to deliver the potential to muscles and/or nerves of such a standard pelvic structure of a female.

In one exemplary embodiment of this aspect of the present invention, a system may include at least one first body member, the power member of the first type, and the electrode member of the first type. In one example, the first body member may be arranged to form a shape of an elongated tubing which includes an annular side and a pair of opposing ends one of which is open while the other of which is closed, to enclose a tip of a penile structure of the male by the closed end, and then to wrap around at least a portion of a shaft of the penile structure with the side. In another example, the first body member may be arranged to define a shape of an elongated tubing having an annular side and a pair of opposing open ends, and to wrap around at least a portion of a shaft of a penile structure of the male while leaving a tip of the penile structure exposed through one of the open ends. In another example, the first body member may be arranged to define a shape of a sheet with a pair of opposing edges capable of releasably coupling onto each other and to wrap around at least a portion of a shaft of a penile structure of the male when the edges are coupled to each other.

In another exemplary embodiment of this aspect of the present invention, a system may include at least one second body member, the power member of the first type, and the electrode member of the second type. In one example, the second body member may be arranged to define a shape of an elongated and expanding funnel having an annular side and a pair of opposing open ends, and to be inserted into the internal cavity. In another example, such a second body member may be arranged to have a shape of an annular ring with an opening therein, and to be disposed around the orifice while aligning the opening with the orifice.

In another aspect of the present invention, a stimulation system may also be provided to deliver electric potential to muscles and/or nerves of the standard pelvic structure of a female.

In one exemplary embodiment of this aspect of the present invention, such a system may have at least one first body member and at least one electrode member. Such a first body member may be arranged to define an elongated and annular tubing having an exterior surface and an interior surface and defining an internal space therein, to define a pair of opposing ends at least one of which is open, to be electrically insulative, and to receive at least a portion of a penile structure of a male into such an internal space. In one example, the electrode member may be arranged to be disposed on the exterior surface of the first body member, to be electrically conductive, and to extend a preset length along the exterior surface, thereby conducting electric potential from one end to another end thereof therealong while electrically insulating the internal space from such potential. In another example, the electrode member may be arranged to extend from the exterior surface to the interior surface of the first body member, to be electrically conductive, and then to extend a preset length along the exterior surface, thereby conducting electric potential from one end to another end thereof therealong while conducting the potential onto the interior surface as well.

In another exemplary embodiment of this aspect of the present invention, a system may include at least one first body member and at least one electrode member. Such a first body member may be arranged to define an elongated and annular tubing having an exterior surface and an interior surface and defining an internal space therein, to define a pair of opposing ends at least one of which is open, and to receive into the internal space at least a portion of a penile structure of a male, where at least a part of the first body member may be arranged to be electrically conductive. The electrode member may be disposed on the exterior surface of such a first body member, and arranged to be electrically conductive, and to extend a preset length along the exterior surface while also electrically contacting the electrically conductive part of the first body member, thereby conducting electric potential from one end to another end thereof therealong while conducting the potential onto the interior surface as well.

In another aspect of the present invention, such a stimulation system may also be arranged to provide electric potential in order to stimulate nerves and/or to contract muscles of the standard pelvic structure.

In one exemplary embodiment of this aspect of the present invention, a system may include the body member of the third type, at least one power member, the electrode member of the third type, the control member, and so on. The power member may be arranged to store therein energy for multiple potentials. The control member may be arranged to operatively couple with the power and electrode members, to generate the potential by drawing only a part of the energy from the power member per a single contraction of the muscles and/or stimulation of the nerves, and to deliver the potential through the electrode member to at least one of such structures when the body member of the third type may engage at least one of the pelvic and penile structures, thereby causing contraction of the muscles of the pelvic structure and/or stimulation of the nerves thereof.

In another exemplary embodiment of this aspect of the present invention, a system may include the body member of the third type, at least one power member, the electrode member of the third type, and at least one control member. The power member may be arranged to include at least one movable part capable of moving with movement of the body member of the third type and then to convert such movement into the potential. Such a control member may be arranged to be operatively coupled to the power and electrode members and to deliver such potential generated from the movement to at least one of the structures through the electrode member, thereby causing contraction of the muscles of the pelvic structure and/or stimulation of the nerves thereof.

In another exemplary embodiment of this aspect of the present invention, a system may include the body member of the third type, at least one power member, the electrode member of the third type, and at least one control member. The power member may be arranged to be brought into contact with at least one of the structures and to convert energy associated with such a contact into the potential. The control member may be arranged to be operatively coupled to the power and electrode members and to deliver the potential generated through the contact to at least one of the structures through the electrode member, thereby resulting in the contraction of such muscles of the pelvic structure and/or stimulation of the nerves thereof.

In another exemplary embodiment of this aspect of the present invention, a system may include the body member of the third type, at least one power member, the electrode member of the third type, and at least one control member. The power member may be arranged to have an electric conductor and/or a magnet capable of moving with movement of such a body member of the third type, to induce electromagnetic induction when moving through external magnetic and electric fields, and to generate the potential therethrough when moving along with the body member of the third type through at least one of such fields. The control member may be arranged to be operatively coupled to the power and electrode members, and to deliver the potential generated by the power member by the induction to at least one of the structures by the electrode member, thereby causing contraction of such muscles of the pelvic structure and/or stimulation of the nerves thereof.

In another aspect of the present invention, a stimulation system may be provided to generate electric potential by movement of at least one part thereof and to stimulate nerves and/or to contract muscles of the standard pelvic structure of a female.

In one exemplary embodiment of this aspect of the present invention, a system may include the body member of the third type, at least one power member, the electrode member of the third type, at least one control member, and so on. The power member may include at least one movable unit and at least one convert unit and may be disposed in the body member of the third type, where the movable unit may be arranged to move along with at least one of the members, while the convert unit may be arranged to be operatively coupled to the movable unit and to convert movement of the movable unit into the potential. The control member may be arranged to be operatively coupled to such power and electrode members and then to deliver such potential generated from the movement to at least one of the structures through the electrode member, thereby causing contraction of the muscles of the pelvic structure and/or stimulation of the nerves thereof.

In another exemplary embodiment of this aspect of the present invention, a system may include the body member of the third type, at least one power member, the electrode member of the third type, and at least one control member. The power member may be arranged to include at least one movable unit and at least one convert unit, where the movable unit may be arranged to be releasably coupled to at least one of the structures and to move along with at least one of such structures, while such a convert unit may be arranged to operatively couple with the movable unit so as to convert movement of the movable unit into the potential. The control member may be arranged to be operatively coupled to the power and electrode members and to deliver the potential generated from the movement of the movable part to at least one of the structures by the electrode member, thereby causing contraction of the muscles of the pelvic structure and/or stimulation of the nerves thereof.

In another exemplary embodiment of this aspect of the present invention, a system may include the body member of the third type, at least one power member, the electrode member of the third type, and at least one control member. The power member may be arranged to include at least one movable unit and at least one convert unit, where the movable unit may be arranged to releasably couple with a body portion of the female and/or a male and to move along with the body portion, and where such a convert unit may be arranged to operatively couple with the movable unit so as to convert movement of the body portion into the potential. The control member may be arranged to be operatively coupled to the power and electrode members and to deliver the potential generated from the movement of the body portion to at least one of the structures by the electrode member, thereby causing contraction of the muscles of the pelvic structure and/or stimulation of the nerves thereof.

In another aspect of the present invention, a stimulation system may be provided to generate electric potential by receiving contact energy with at least one part thereof and capable of stimulating nerves and/or contracting muscles of the standard pelvic structure.

In one exemplary embodiment of this aspect of the present invention, a system may include the body member of the third type, at least one power member, the electrode member of the third type, at least one control member, and so on. In one example, the power member may be arranged to have at least one part capable of forming a contact with at least one of the structures while absorbing at least a portion of the energy which is associated with the contact, and to convert the absorbed energy into the potential. In yet another example, the power member may be arranged to include at least one part capable of varying its configuration while contacting at least one of the structures and absorbing at least a portion of the energy accompanying the contacting and to convert the absorbed energy into the potential. In another example, the power member may also be arranged to have at least two parts capable of moving toward (or away from) each other while contacting at least one of the structures and while absorbing at least a portion of the energy accompanying the contacting and to convert the absorbed energy into the potential. In each of these examples, the control member may be arranged to be operatively coupled to the power and electrode members and to deliver the potential generated by the power member to at least one of such structures by the electrode member, thereby resulting in contraction of the muscles of the pelvic structure and/or stimulation of the nerves thereof.

In another exemplary embodiment of this aspect of the present invention, a system may include the body member of the third type, at least one power member, the electrode member of the third type, and at least one control member. In one example, such a power member may be arranged to include at least one part capable of forming a contact with at least one body portion of the female and/or male while absorbing at least a portion of the energy associated with the contact, and then to convert such absorbed energy into the potential. In another example, the power member may be arranged to have at least one part capable of varying its configuration while contacting at least one body portion of the female and/or a male and absorbing at least a portion of the energy accompanying the contacting and to convert the absorbed energy into the potential. In yet another example, the power member may be arranged to include at least two parts which may be capable of moving toward (or away from) each other while contacting at least one body portion of the female and/or a male and absorbing at least a portion of the energy accompanying the contacting and then to convert the absorbed energy into the potential. In each of these examples, the control member may be arranged to be operatively coupled to the power and electrode members and to deliver the potential generated by contacting the power member by such at least one body portion to at least one of such structures by the electrode member, thereby causing contraction of the muscles of the pelvic structure and/or stimulation of the nerves of such a structure.

In another aspect of the present invention, a stimulation system may be arranged to provide electric potential by electromagnetic induction and to deliver the potential to muscles and/or nerves of the standard pelvic structure of a female.

In one exemplary embodiment of this aspect of the present invention, a system may include the body member of the third type, at least one power member, the electrode member of the third type, at least one control member, and the like. The power member may include at least one internal field unit and at least one movable unit, where the internal field unit may be arranged to generate therearound an electric field and/or magnetic field, where the movable unit may be arranged to move relative to the internal field unit and to include therein a magnet and/or an electric conductor, while the movable unit may be arranged to be disposed in the body member of the third type. Such a control member may be arranged to operatively couple with the electrode and power members and to deliver to at least one of the structures through the electrode member the potential which may be generated by electromagnetic induction when the movable unit generates movement with respect to the internal field unit, thereby causing contraction of the muscles of the pelvic structure and/or stimulation of the nerves thereof.

In another exemplary embodiment of this aspect of the present invention, a system may include the body member of the third type, at least one power member, the electrode member of the third type, and at least one control member. The power member may be arranged to include at least one internal field unit and at least one movable unit, where the internal field unit may be incorporated into the body member of the third type and to generate therearound an electric field and/or magnetic field, while the movable unit may be arranged to be incorporated into the other of the body member of third type and to include an electric conductor and/or a magnet. The control member may be arranged to operatively couple with the electrode and power members and to deliver by the electrode member such potential which is generated by electromagnetic induction when the first (or second) body member generates movement with respect to the second (or first) body member, thereby resulting in contraction of such muscles of the pelvic structure and/or stimulation of the nerves thereof.

In another exemplary embodiment of this aspect of the present invention, a system may include the body member of the third type, at least one power member, the electrode member of the third type, and at least one control member. The power member with at least one movable unit may be arranged to have therein a magnet and/or an electric conductor and to be disposed in the body member of the third type. The control member may be arranged to operatively couple with the electrode and power members and to deliver to at least one of the structures through the electrode member such potential which is generated by electromagnetic induction when the movable unit generates movement across an external electric field and/or external magnetic field, thereby causing contraction of the muscles of the pelvic structure and/or stimulation of the nerves thereof.

In another aspect of the present invention, a stimulation system may be arranged to provide electric potential in order to stimulate nerves and/or contract muscles of the standard pelvic structure of a female.

In one exemplary embodiment of this aspect of the present invention, a system may include the body member of the third type, the power member of the first type, the electrode member of the third type, and at least one control member which may be arranged to operatively couple with the electrode and power members and to also include at least one sensor unit and at least one control unit. In one example, the sensor unit may be arranged to measure at least one variable including at least one of a displacement of the body member of the third type, its velocity, its acceleration, a direction of at least one of such displacement, velocity, and acceleration, a duration (or period) thereof, and a frequency thereof. The control unit may be arranged to deliver such potential from the power member to at least one of the structures through the electrode member when a value of the variable exceeds beyond a preset value, thereby causing contraction of the muscles of the pelvic structure and/or stimulation of the nerves of such a structure. In another example, the sensor unit may be arranged to measure at least one variable including at least one of a force of its contact with at least one of the structures, a position of such contact, a duration thereof, and a frequency thereof, and where the control unit may be arranged to deliver the potential from the power member to at least one of the structures through the electrode member when a value of the variable exceeds beyond a preset value, thereby causing contraction of the muscles of the pelvic structure and/or stimulation of the nerves thereof. In another example, the sensor unit may be arranged to measure at least one variable including at least one of a insertion depth thereof along the internal cavity, a distance to a preset reference point therefrom, its orientation with respect to such a reference point, a direction to the reference point therefrom, and an elevation above (or below) the reference point, and where the control unit may be arranged to deliver the potential from the power member to at least one of the structures by the electrode member when a value of the variable may exceed a preset value (or falls within a preset range), thereby resulting in contraction of the muscles of the pelvic structure and/or stimulation of the nerves thereof.

In another exemplary embodiment of this aspect of the present invention, a system may include the body member of the third type, at least one power member, the electrode member of the third type, and a control member. The power member may be arranged to provide power for such potential in at least a substantial synchronization with at least one variable of the system, pelvic structure, and/or penile structure. The control member may be arranged to be operatively coupled to the electrode and power members and to include at least one sensor unit and at least one control unit. In one example, the sensor unit may be arranged to measure at least one variable which may include at least one of a displacement of the body member of the third type, its velocity, its acceleration, a direction of at least one of such displacement, velocity, and acceleration, a duration (or period) thereof, and a frequency thereof, and where the control unit may be arranged to deliver the potential from the power member to at least one of such structures by the electrode member in synchronization with at least one dynamic pattern of such a variable, thereby causing contraction of such muscles of the pelvic structure and/or stimulation of the nerves thereof. In another example, the sensor unit may be arranged to measure at least one variable including at least one of a force of its contact with at least one of such structures, a position of the contact, a duration thereof, and a frequency thereof, and where the control unit may be arranged to deliver the potential from the power member to at least one of such structures through the electrode member in synchronization with at least one dynamic pattern of such a variable, thereby causing contraction of the muscles of the pelvic structure and/or stimulation of the nerves thereof. In yet another example, the sensor unit may be arranged to sense at least one variable including at least one of a insertion depth thereof along such an internal cavity, a distance to a preset reference point therefrom, its orientation with respect to such a reference point, a direction toward such a reference point therefrom, and an elevation above (or below) the reference point, while the control unit may be arranged to deliver the potential from the power member to at least one of the structures through the electrode member in synchronization with at least one dynamic pattern of such a variable, thereby causing at least one of contraction of the muscles and stimulation of the nerves.

In another aspect of the present invention, a stimulation system may be arranged to provide electric potential and delivering the potential to muscles and/or nerves of the standard pelvic structure of a female, where the potential may be arranged to contract the muscles of the pelvic structure in a preset order.

In one exemplary embodiment of this aspect of the present invention, a system may include the body member of the first type, the power member of the first type, and at least one electrode member. The electrode member may be disposed on the first body member in an orientation matching that of the pelvic structure and arranged to receive the potential from the power member and then to deliver such potential to the pelvic structure therethrough as the first body member engages the pelvic structure, thereby causing contraction of the muscles of the pelvic structure and/or stimulation of the nerves of such a structure in an uniform direction.

In another exemplary embodiment of this aspect of the present invention, such a system may have the body member of the first type, the power member of the first type, and at least one electrode member. The electrode member may be disposed on the first body member in an orientation matching that of the pelvic structure, and arranged to receive the potential from the power member, and then to deliver such potential to the pelvic structure therethrough along alternating directions as the first body member may engage the pelvic structure, thereby resulting in contraction of the muscles of the pelvic structure and/or stimulation of the nerves thereof in opposing directions alternatingly.

In another exemplary embodiment of this aspect of the present invention, a system may include the body member of the first type, the power member of the first type, and multiple electrode members which may be arranged to be incorporated into the first body member, to receive the potential from the power member, and to deliver such potential to the pelvic structure therethrough when the first body member engages the pelvic structure in one of multiple directions, thereby causing contraction of such muscles of the pelvic structure and/or stimulation of the nerves thereof in the directions.

In another exemplary embodiment of this aspect of the present invention, such a system may also include the body member of the first type, the power member of the first type, multiple electrode members, and at least one control member. Multiple electrode members may be incorporated into the first body member and arranged to receive the potential from the power member. The control member may be arranged to operatively couple with such electrode and power members, to measure at least one variable of at least one of the system and pelvic structure, and to deliver to at least one of such structures through the electrode member the potential along a direction which is in turn determined by a value of the measured variable, thereby causing contraction of the muscles of the pelvic structure and/or stimulation of the nerves thereof depending upon the value of the variable.

Embodiments of such apparatus aspects of the present invention may include one or more of the following features.

The first body member may preferably be designed to engage with the clitoris, G spot, and/or other portions of the pelvic structure of the female. The second body member may be arranged to be inserted into the internal cavity of the pelvic structure and/or to be disposed around the orifice thereof. The body member of the first type of any of the above systems may be replaced by the body member of the third type, whereas the body member of the second type of any of the above systems may be replaced by the body member of the third type. However, the body member of the third type of any of the above systems may be replaced by the body member of first type and/or that of the second type, depending on its disposition. Such a body member may be coated with a liquid, solution, suspension, cream, powder, and/or gel which may be electrically conductive and used for sanitary, storage, use, contraception, and/or conduction purposes. The body member may be coated with a liquid, solution, suspension, cream, powder, and/or gel which may be electrically insulative and employed for storage, use, contraception, sanitary, and/or insulation purposes. The portion of the pelvic structure may be a clitoris of the entry, a G spot on the wall, other portions of the wall, and the like.

The power member may be disposed on the body member or, alternatively, the power member may be disposed away from the body member. In addition, the power member may not be a member of the system but a member external thereto. The system may include at least three modules at least two of which may include at least one of the members. Such an external device may be an external audiovisual device, an external communication device, and/or an internet.

The electrode member may electrically contact a point portion of the pelvic structure, an area portion thereof, and the like, when the first body member engages the pelvic structure. The electrode member may electrically contact a point portion of the penile structure, an area portion thereof, and the like, when the second body member may engage such a penile structure. The electrode member may electrically contact both of such pelvic and penile structures. At least a part of the electrode member may extend along an axial direction of the body member, a circumferential direction thereof, a radial direction thereof, and/or in a helical direction thereof. The system may also include multiple electrode members, at least two of which may conduct the potential in a series mode, in a parallel mode, and/or individually without any coupling therebetween.

The body portion may include at least one of a finger, a hand, a wrist, an arm, a toe, a foot, a leg, a thigh, a waist, an abdomen, a back, an upper torso, a shoulder, a neck, and a head of the at least one of the female and male. The power member may include multiple movable parts disposed in different body portions of one of the female and male. The power member may also include multiple movable parts at least one of which may be disposed in at least one of the body portions of one of the female and male and at least another of which may be disposed in at least one of the body portions of the other of such a female and male. The power member may include at least one electromechanical generator for converting such movement and/or contact into the potential, at least one piezoelectric generator for converting such abutting (or force associated therewith) into the potential, and the like. The field may be a static electric, magnetic or electromagnetic field or dynamic (or alternating) electric, magnetic or electromagnetic field. Such an external field may be formed by a single magnet emanating concentric magnetic field lines. The external field may instead be formed by multiple magnets capable of defining at least substantially linear magnetic field lines therebetween. Such a power member may include at least one capacitor capable of storing the potential therein and to deliver the potential only when an amplitude of the stored potential exceed a preset value.

Such a variable may be at least one of a normal force exerted by the portion of the structure, a bending force exerted thereby, an axial force exerted thereby to pull (or push) an object (or the body member) into (or out of) the internal cavity, torque exerted thereby, contact between the object (or the body member) and the portion of the structure, and the like. Such a variable may be at least one of a normal force applied to at least a part of the body member, a bending force applied onto the part, an axial force pulling (or pushing) the part (or object) into (or out of) the internal cavity, a torque which may be applied around (or with respect to) the part, a velocity of such a part, an acceleration thereof, a displacement thereof, a contact between the part and portion of the structure, a dimension of such a portion, contraction and relaxation of the portion, a duration of at least one of the above variables, a frequency of at least one of the variables, and the like. The variable may be at least one of a normal force applied onto the portion of the structure, a bending force applied onto the portion, an axial force resisting movement of an object (or the body member) into (or out of) such a cavity of the structure, a torque applied about such a portion, a velocity of the portion, an acceleration thereof, a displacement thereof, a contact between the portion and body member, contraction and relaxation of the portion, a duration of at least one of the variables, a frequency of at least one of the variables, and so on.

The variable may define the dynamic pattern which may include at least one temporal pattern and at least one spatial pattern, and such a sensor unit may be arranged to measure (or monitor) the temporal and/or spatial patterns. The temporal pattern may be at least one of an instantaneous value of the variable, its time-varying value, its time-averaged value, its average which may be weighted by a preset weighting function, its peak value, its time derivative, its integration over time, and so on. The temporal pattern may be at least one of a duration of such a variable, its frequency, and its temporal sequence. Such a spatial pattern may be at least one of a localized value of the variable in a preset region, a distribution of multiple variables over an area, a space-averaged value of the variable, its global or local peak, its derivative along a preset direction, its integration in at least one direction, and the like. The spatial pattern may include at least one of an amplitude of the variable and its direction. The dynamic pattern may be at least one of a frequency of the variable, its temporal rate of change or its temporal differentiation, its displacement (or its integral over time) caused thereby, and a compound value obtained through at least one of mathematical manipulation of at least one thereof. The variable and/or value may be in an analog or digital format.

Such a control member may obtain one of the variables and assess at least one another of the variables from the one of the variables. The sensor unit may measure the variable along any direction with respect to a longitudinal axis of the body member. The sensor unit may measure at least one of an absolute value of the variable and a relative (or normalized) value of the variable with respect to a preset value thereof. The control member may have any number of such sensor units which may be disposed in any arrangements and each of which may measure any of the above variables. Such a body member may include multiple sensor units which may be identical, disposed close to each other, disposed apart from each other, and the like.

The control member may be synchronized not only to deliver potentials to the pelvic structure but also to manipulate various dynamic features of such potentials. For example, the control member may manipulate a value (i.e., an amplitude) of the potential delivered to the pelvic structure, a duration thereof, values of multiple potential, intervals between such potentials, and the like. In other words, such potential may also define such dynamic feature which may include at least one temporal feature and at least one spatial feature, and such a system may generate and deliver the potential with such temporal and/or spatial features. The temporal feature may be at least one of an instantaneous value of the potential, its time-varying value, its time-averaged value, its average which may be weighted by a preset weighting function, its peak value, its time derivative, its integration over time, and so on. The temporal feature may be at least one of a duration of such potential, its frequency, and its temporal sequence. Such a spatial pattern may be at least one of a localized value of such potential in a preset region, a distribution of multiple potentials over an area, a space-averaged value of such potential, its global or local peak, its derivative along a preset direction, its integration in at least one direction, and the like. The spatial feature may include at least one of an amplitude of the potential and its direction. The dynamic feature may be at least one of a frequency of the potential, its temporal rate of change or its temporal differentiation, its displacement (or its integral over time) caused thereby, and a compound value obtained through at least one of mathematical manipulation of at least one thereof. The potential may be provided in an analog or digital format.

The system may play sound and/or display an image of the variable and/or value with internal audio, visual, and/or audiovisual input and/or output units or with external audio, visual or audiovisual input and/or output devices. The sound and/or image may also be generated in response to the value and/or variable effected or initiated by the user or may instead be generated to effect the value and/or variable by the user. The system may include at least one of an audio output unit capable of playing the value as audible signals and a visual output unit capable of displaying the value as visual signals.

In another aspect, a method may be provided for contracting muscles of the standard pelvic structure and/or stimulating nerves of such a structure of a female with electric potential.

In one exemplary embodiment of this aspect of the invention, such a method may include the steps of: disposing at least one conductive electrode member on a first body member (to be referred to as the “first disposing” hereinafter); placing the first body member on at least a portion of a penile structure of a male (to be referred to as the “placing” hereinafter); and then engaging the first body member with at least a portion of the pelvic structure while providing the potential thereto, thereby causing contraction of the muscles and/or stimulation of the nerves thereof.

In another exemplary embodiment of this aspect of the invention, a method may have the steps of: disposing at least one conductive electrode member on a second body member (to be referred to as the “second disposing” hereinafter); positioning the second body member on or in at least a portion of the pelvic structure (to be referred to as the “positioning” hereinafter); and then receiving into such a second body member at least a portion of a penile structure of a male while providing such potential onto the pelvic structure, thereby attaining contraction of the muscles and/or stimulation of the nerves.

In another exemplary embodiment of such an aspect of the invention, a method may include the steps of: disposing at least one conductive electrode member onto at least one of a first body member and a second body member (to be referred to as the “third disposing” hereinafter); the above placing; the above positioning; and then engaging the first body member with the second body member while providing the potential to such a pelvic structure, thereby causing contraction of the muscles and/or stimulation of the nerves.

In another exemplary embodiment of such an aspect of the invention, a method may include the steps of: the first disposing; placing the first body member on at least a portion of an object; and then engaging such an object with the pelvic structure while providing the potential to the pelvic structure, thereby causing contraction of the muscles and/or stimulation of the nerves.

In another exemplary embodiment of such an aspect of the invention, a method may include the steps of: engaging at least one conductive body member with the pelvic structure; the first disposing; and engaging the first body member with the pelvic structure while providing the potential to the pelvic structure, thereby attaining contraction of the muscles and/or stimulation of the nerves.

In another aspect, a method may be provided for contracting muscles of the standard pelvic structure and/or stimulating nerves of such a structure of a female with electric potential provided by at least one of multiple modules.

In one exemplary embodiment of this aspect of the invention, a method may include the steps of: the first disposing; providing at least one power member which is capable of providing power for the potential (to be referred as the “providing” hereinafter); incorporating at least one of the members into a first module; incorporating the rest of the members into a second module; operatively coupling the first and second modules; placing one of the modules including therein the first body member on at least a portion of a penile structure of a male; and then engaging the structures with each other while providing the potential to such a pelvic structure, thereby causing contraction of the muscles and/or stimulation of the nerves thereof.

In another exemplary embodiment of such an aspect of the invention, a method may include the steps of: the second disposing; the above providing; incorporating at least one of the members into a first module; incorporating the rest of the members into a second module; operatively coupling the first and second modules; positioning one of the modules including therein the second body member on at least a portion of the pelvic structure; and engaging the structures with each other while providing the potential to the pelvic structure, thereby causing contraction of the muscles and/or stimulation of the nerves thereof.

In another exemplary embodiment of such an aspect of the invention, a method may include the steps of: the third disposing; the above providing; incorporating at least one of the members into a first module; then incorporating the rest of the members into a second module; placing one of the modules including the first body member on at least a portion of a penile structure of a male; positioning one of the modules having therein the second body member on at least a portion of the pelvic structure; and engaging the modules with each other while providing such potential to the pelvic structure, thereby causing contraction of the muscles and/or stimulation of the nerves thereof.

In another aspect, a method may be provided for contracting muscles of the standard pelvic structure and/or stimulating nerves of such a structure of a female by electric potential in cooperation with at least one external device.

In one exemplary embodiment of this aspect of the invention, a method may have the steps of: the third disposing, the above providing; receiving at least one signal from the external device; placing or positioning such at least one of the body members on at least a portion of at least one of the pelvic structure and a penile structure of a male; and engaging such at least one of the body members with at least one of the structures while providing the potential to the pelvic structure based on the external signal, thereby causing contraction of the muscles and/or stimulation of the nerves thereof.

In another exemplary embodiment of such an aspect of the invention, a method may include the steps of: the third disposing; receiving power for the potential from such an external device; placing or positioning such at least one of the body members on at least a portion of at least one of the pelvic structure and a penile structure of a male; and engaging such at least one of the body members with at least one of the structures while providing such potential to the pelvic structure, thereby attaining contraction of the muscles and stimulation of the nerves.

In another aspect, a method may be provided for contracting muscles of the standard pelvic structure and/or stimulating nerves of the pelvic structure of a female by electric potential provided by one of the female and a male.

In one exemplary embodiment of this aspect of the invention, a method may include the steps of: elongating at least one annular first body member to form a cylindrical side; opening one end of such a first body member while closing the other end thereof; the first disposing; enclosing a tip of a penile structure of the male with the closed end while wrapping around at least a portion of a shaft of the penile structure with the side; and engaging the first body member with at least a portion of such a pelvic structure while providing the potential to the pelvic structure, thereby causing contraction of the muscles and/or stimulation of the nerves thereof. The above steps from elongating to enclosing may be replaced by the steps of: elongating at least one annular first body member to define a cylindrical side; opening a pair of opposing ends of the first body member; the first disposing; and wrapping around at least a portion of a shaft of a penile structure of the male with the first body member while leaving a tip of the penile structure exposed through one of the ends. Alternatively, the above steps from elongating to enclosing may also be replaced by the steps of: spreading at least one first body member into a sheet; the first disposing; and wrapping around at least a portion of a shaft of a penile structure of the male while coupling a pair of opposing edges of the first body member.

In another exemplary embodiment of such an aspect of the invention, a method may include the steps of: elongating at least one annular second body member to define a cylindrical side; opening a pair of opposing ends of the second body member; the second disposing; inserting such a second body member with at least a portion of the pelvic structure; and engaging a penile structure of a male with the second body member while providing the electric potential onto the pelvic structure, thereby accomplishing contraction of the muscles and/or stimulation of the nerves thereof. The above steps of elongating, opening, and disposing may be replaced by the steps of: elongating at least one annular second body member to form a cylindrical ring; opening a pair of opposing ends of the second body member; and the second disposing.

In another aspect, a method may be provided for contracting muscles of the standard pelvic structure and/or stimulating nerves of the pelvic structure of a female with electric potential.

In one exemplary embodiment of this aspect of the invention, a method may include the steps of: elongating an annular first body member with a cylindrical and electrically insulative side; defining on the side an exterior surface and an interior surface; extending at least one conductive electrode member on the exterior surface of the first body member; the above placing; and engaging such a first body member with at least a portion of the pelvic structure while providing such potential to the pelvic structure along the electrode member, thereby causing contraction of the muscles and/or stimulation of the nerves thereof while insulating the penile structure from such potential. The above extending, placing, and engaging may be replaced by the steps of: extending at least one conductive electrode member on the exterior surface of the first body member and across the side onto the interior surface of the first body member; the above placing; and then engaging the first body member with at least a portion of the pelvic structure while providing the potential to the pelvic structure along the electrode member, thereby causing contraction of the muscles of the pelvic structure and/or stimulation of the nerves thereof as well as contraction of muscles of the penile structure and/or stimulation of nerves of the penile structure.

In another exemplary embodiment of such an aspect of the invention, a method may include the steps of: elongating an annular first body member with a cylindrical and electrically conductive side; defining an exterior surface and an interior surface along the side; extending at least one conductive electrode member on the exterior surface of the first body member; the above placing; and engaging the first body member with at least a portion of the pelvic structure while providing the potential to the pelvic structure along the electrode member, thereby causing contraction of the muscles of the pelvic structure and/or stimulation of such nerves thereof and causing contraction of muscles of the penile structure and/or stimulation of nerves of the penile structure.

In another aspect, a method may be provided for providing electric potential for the purpose of contracting muscles of the standard pelvic structure and/or stimulating nerves of such a structure of a female.

In one exemplary embodiment of this aspect of the invention, a method may include the steps of: the third disposing; storing energy in an amount enough to generate multiple the potentials; placing or positioning such at least one of the body members on at least a portion of at least one of the pelvic structure and a penile structure of a male; engaging such at least one of the body members with at least one of the structures; and drawing only a part of the energy, thereby providing the potential to the pelvic structure and causing contraction of the muscles and/or stimulation of the nerves thereof per each of the drawing.

In another exemplary embodiment of such an aspect of the invention, a method may include the steps of: the third disposing; providing at least one power member capable of generating the potential by its movement; placing or positioning such at least one of the body members on at least a portion of at least one of the pelvic structure and a penile structure of a male; engaging such at least one of the body members with at least one of the structures; and moving the power member, thereby providing the potential to the pelvic structure as well as causing contraction of the muscles and/or stimulation of the nerves.

In another exemplary embodiment of such an aspect of the invention, a method may include the steps of: the third disposing; providing at least one power member capable of generating the potential by contact force; placing or positioning such at least one of the body members on at least a portion of at least one of the pelvic structure and a penile structure of a male; engaging such at least one of the body members with at least one of the structures; and applying such force onto the power member, thereby providing the potential onto the pelvic structure and causing contraction of the muscles and/or stimulation of the nerves.

In another exemplary embodiment of such an aspect of the invention, a method may include the steps of: the third disposing; providing at least one power member capable of generating the potential by electromagnetic induction; placing or positioning such at least one of the body members on at least a portion of at least one of the pelvic structure and a penile structure of a male; engaging such at least one of the body members with at least one of the structures; and moving the power member across at least one of magnetic field and electric fields, thereby providing the potential to the pelvic structure and causing contraction of the muscles and/or stimulation of the nerves.

In another aspect, a method may be provided for providing electric potential only upon preset occasions for stimulating nerves of the standard pelvic structure and/or stimulating muscles of such a structure of a female.

In one exemplary embodiment of this aspect of the invention, such a method may include the steps of: the third disposing; placing or positioning such at least one of the body members on at least a portion of at least one of the pelvic structure and a penile structure of a male; engaging such at least one of the body members with at least one of the structures; measuring at least one variable including a displacement of such at least one of the body members, its speed, its acceleration, a direction of the displacement, speed, and/or acceleration, a duration (or period) thereof, and/or a frequency thereof; and providing the potential to the pelvic structure when a value of the variable may exceed a preset value, thereby causing at least one of contraction of the muscles and stimulation of the nerves. The measuring may be replaced by the step of: measuring at least one variable which includes a force of its contact with at least one of the structures, a position of the contact, a duration thereof, and/or a frequency thereof. Such measuring may instead be replaced by the step of: measuring at least one variable including its insertion depth along the internal cavity, a distance to a preset reference point therefrom, its orientation relative to the reference point, a direction to the reference point therefrom, and/or an elevation above (or below) the reference point.

In another exemplary embodiment of such an aspect of the invention, a method may include the steps of: the third disposing; placing or positioning such at least one of the body members on at least a portion of at least one of the pelvic structure and a penile structure of a male; engaging the at least one of the body members with at least one of the structures; and causing contraction of the muscles and/or stimulation of the nerves by providing such potential to the pelvic structure in synchronization with at least one variable which includes a displacement of such at least one of the body members, its acceleration, its velocity, a direction of the displacement, velocity, and/or acceleration, a duration or a period thereof, and/or a frequency thereof. In another example, such a variable may include a force exerted by its contact with at least one of the structures, a position of the contact, a duration thereof, and/or a frequency thereof. In yet another example, such a variable may include an insertion depth of such at least one of the members along such an internal cavity, a distance to a preset reference point therefrom, its orientation relative to the reference point, a direction to the reference point therefrom, and/or an elevation above (or below) the reference point.

In another aspect, a method may be provided for contracting muscles of the standard pelvic structure and/or stimulating nerves of such a structure of a female by electric potential according to a preset order.

In one exemplary embodiment of this aspect of the invention, a method may include the steps of: the first disposing; the above placing; and engaging the first body member with at least a portion of the pelvic structure while providing the potential to the pelvic structure in an uniform direction through the electrode member, thereby attaining contraction of such muscles and/or stimulation of the nerves thereof in another uniform direction. The engaging may be replaced by the step of: engaging the first body member with at least a portion of the pelvic structure while providing such potential to the pelvic structure in alternating directions through the electrode member, thereby causing contraction of such muscles and/or stimulation of the nerves thereof in opposite directions.

In another exemplary embodiment of such an aspect of the invention, a method may include the steps of: disposing multiple conductive electrode members on a first body member; the above placing; and engaging the first body member with at least a portion of the pelvic structure while providing such potential to the pelvic structure in each of multiple directions by the electrode member, thereby causing contraction of the muscles and/or stimulation of the nerves thereof in each of multiple directions.

In another exemplary embodiment of such an aspect of the invention, a method may include the steps of: the first disposing; the above placing; and then engaging the first body member with at least a portion of the pelvic structure while providing the potential onto the pelvic structure through such an electrode member along a direction which is determined by at least one variable of the pelvic and/or penile structures, thereby causing contraction of the muscles and/or stimulation of the nerves thereof in the direction.

Embodiments of such method aspects of the present invention may include one or more of the following features.

The disposing may include at least one of the steps of: forming at least one point portion of the electrode member; and defining an area portion thereof, where the defining may include the step of: extending the area portion of the electrode member at least one of axially, radially, angularly, and/or helically. The disposing may include the step of: disposing the electrode member onto at least one of an exterior surface of the body member, an interior surface thereof, and across the body member. The disposing may include the step of: exposing the electrode member on a surface of such a body member which is to electrically contact only the pelvic structure, only the penile structure, or both of the pelvic and penile structures. The disposing may include the steps of: disposing multiple electrode members to the body member; and coupling at least two of the electrode members in a series and/or parallel mode. The disposing may include the steps of: disposing multiple electrode members onto the body member; and supplying the potential through the electrode members individually.

The placing may include one of the steps of: enclosing the tip of the penile structure by such a first body member; and exposing the tip of the penile structure. The positioning may include at least one of the steps of: inserting the second body member in the internal cavity; and disposing the second body member around the orifice. The positioning may include the step of: contacting at least one of a clitoris of the entry, a G spot on the wall, and other portions of the wall with the second body member. The incorporating may include the steps of: providing at least three modules; and operatively coupling the modules for providing the potential.

The providing may include at least one of the steps of: disposing such a power member on the body member; and disposing such a power member away from the body member. The providing may include the step of: providing the power member as an external device. The providing may include the steps of: including at least one capacitor capable of storing such potential therein; and delivering such potential only when an amplitude of the stored potential may exceed a preset value. The moving may include one of the steps of: actuating the power member through the pelvic structure; actuating such a power member with the penile structure; and then actuating the power member by at least one body portion including at least one of a finger, a hand, a wrist, an arm, a toe, a foot, a leg, a thigh, a waist, an abdomen, a back, an upper torso, a shoulder, a neck, a face, and a head of the at least one of the female and male. The moving may include the steps of: including multiple movable units different body portions of one of the female and male; and moving at least one of the movable units The moving may include the steps of: releasably disposing at least one movable unit onto the pelvic structure; and then releasably disposing at least another movable unit onto the penile structure. The providing may have the steps of: including at least one electromechanical generator; and converting movement of at least one body member into the potential. The providing may include the steps of: incorporating at least one piezoelectric generator; and converting contact force applied thereto into the potential. The providing may include the steps of: generating at least one of a static magnetic field and a dynamic or alternating magnetic field; and moving the conductor thereacross. The providing may further include the steps of: generating at least one of a static electric field and a dynamic (or alternating) electric field; and moving the magnet thereacross.

The method may include one of the steps of: coating the body member with a liquid, solution, suspension, cream, powder, and/or gel which may be electrically conductive and used for storage, use, contraception, sanitary, and/or conduction purposes; and coating the body member with a liquid, solution, suspension, cream, powder, and/or gel which may be electrically insulative and employed for storage, use, contraception, sanitary, and/or insulation purposes. The method may include the step of: generating at least one of sounds and images connoting the variable. The generating may include one of the steps of: performing the generating as a response to the variable effected (or initiated) by an user; and performing the generating to effect the variable by the user.

In another aspect of the present invention, a stimulation system may provide electric potential and deliver the potential to muscles and/or nerves of the standard pelvic structure of a female.

In one exemplary embodiment of this aspect of the invention, such a system may be made by a process including the steps of: arranging a first body member to receive at least a portion of a penile structure of a male; providing at least one power member capable of providing power for the potential; disposing at least one electrode member on the first body member; generating the potential with such a power member; and delivering the potential to the pelvic structure by the electrode member as such a first body member engages the pelvic structure, thereby causing contraction of the muscles and/or stimulation of the nerves.

In another exemplary embodiment of this aspect of the invention, such a system may be made by a process including the steps of: arranging a second body member to receive at least a portion of the pelvic structure; providing at least one power member which is capable of providing power for such potential; disposing at least one electrode member on the second body member; generating the potential by the power member; and delivering such potential to the pelvic structure by the electrode member when the second body member engages a penile structure of a male, thereby accomplishing contraction of the muscles and/or stimulation of the nerves.

In another exemplary embodiment of this aspect of the invention, such a system may be made by a process including the steps of: arranging a first body member to receive at least a portion of a penile structure of a male; arranging a second body member to receive at least a portion of the pelvic structure; providing at least one power member capable of providing power for the potential; placing at least one electrode member on at least one of the body members; generating such potential by the power member; and delivering the potential to the pelvic structure by the electrode member when the first body member engages the second body member, thereby resulting in contraction of the muscles and/or stimulation of the nerves.

In another exemplary embodiment of this aspect of the invention, such a system may be made by a process including the steps of: providing an object to be disposed into the internal cavity and/or around the orifice; arranging a first body member to enclose at least a portion of the object; providing at least one power member which is capable of providing power for the potential; disposing at least one electrode member on the first body member; generating the potential by the power member; and delivering such potential to the pelvic structure by the electrode member when the first body member engages such a pelvic structure, thereby causing contraction of the muscles and/or stimulation of the nerves.

In another exemplary embodiment of this aspect of the invention, such a system may be made by a process including the steps of: arranging a first body member to be disposed into the internal cavity and/or around the orifice; providing at least one power member which is capable of providing power for the potential; disposing at least one electrode member on the first body member; generating the potential by the power member; and delivering the potential to the pelvic structure by the electrode member when the first body member engages the pelvic structure, thereby causing contraction of the muscles and/or stimulation of the nerves.

In another aspect of the present invention, a stimulation system may provide electric potential and then deliver the potential to muscles and/or nerves of the standard pelvic structure of a female in cooperation with a device which is external to the system.

In one exemplary embodiment of this aspect of the invention, such a system may be made by a process including the steps of: arranging a first body member capable of receiving at least a portion of a penile structure of a male; arranging a second body member to receive at least a portion of such a pelvic structure; providing at least one power member capable of providing power for the potential; disposing at least one electrode member on at least one of the body members; arranging at least one of the members to be controlled by the external device; obtaining at least one external signal from the external device; generating the potential by the power member; and then delivering the potential to the pelvic structure by the electrode member based upon the external signal when the first body member engages the pelvic structure, thereby accomplishing contraction of the muscles and/or stimulation of the nerves.

In another exemplary embodiment of this aspect of the invention, such a system may be made by a process including the steps of: arranging a first body member to receive at least a portion of a penile structure of a male; arranging a second body member to receive at least a portion of the pelvic structure; disposing at least one electrode member on at least one of such body members; receiving power for the potential from the external device; generating the potential by the power member; and delivering such potential to the pelvic structure by the electrode member when the first body member engages the pelvic structure, thereby accomplishing contraction of the muscles and/or stimulation of the nerves.

In another aspect of the present invention, a stimulation system may provide electric potential in order to stimulate nerves and/or to contract muscles of the standard pelvic structure of a female.

In one exemplary embodiment of this aspect of the invention, such a system may be made by a process including the steps of: arranging a first body member capable of receiving at least a portion of a penile structure of a male; arranging a second body member to receive at least a portion of such a pelvic structure; providing at least one power member for storing energy for multiple the potentials; disposing at least one electrode member on at least one of the body members; drawing a part of the energy, thereby generating the potential by the power member; and delivering the potential to such a pelvic structure with the electrode member when the first body member engages the pelvic structure, thereby causing at least one of contraction of the muscles and stimulation of the nerves.

In another exemplary embodiment of this aspect of the invention, such a system may be made by a process including the steps of: arranging a first body member to receive at least a portion of a penile structure of a male; arranging a second body member to receive at least a portion of the pelvic structure; providing a portable electric generator capable of converting movement of at least one of the body members into the potential; disposing at least one electrode member on at least one of such body members; engaging at least one of the body members with the pelvic structure; generating the movement of such at least one of the body members, thereby generating the potential by the electric generator; and delivering the potential to the pelvic structure through the electrode member, thereby causing contraction of the muscles and/or stimulation of the nerves.

In another exemplary embodiment of this aspect of the invention, such a system may be made by a process including the steps of: arranging a first body member to receive at least a portion of a penile structure of a male; arranging a second body member to receive at least a portion of the pelvic structure; providing a portable electric generator capable of converting force applied thereonto into the potential; disposing at least one electrode member on at least one of the body members; engaging at least one of the body members with the pelvic structure; applying the contact force onto the electric generator, thereby generating the potential thereby; and delivering the potential to the pelvic structure by the electrode member, thereby causing contraction of the muscles and/or stimulation of the nerves.

In another exemplary embodiment of this aspect of the invention, such a system may be made by a process including the steps of: arranging a first body member to receive at least a portion of a penile structure of a male; arranging a second body member to receive at least a portion of the pelvic structure; providing a portable electric generator capable of producing the potential during movement of at least one of such body members by electromagnetic induction; disposing at least one electrode member on at least one of the body members; supplying electric field and/or magnetic field around the electric generator; engaging at least one of the body members with the pelvic structure; generating the movement of such at least one of the body members, thereby generating the potential by such an electric generator; and delivering the potential to the pelvic structure by the electrode member, thereby causing contraction of the muscles and/or stimulation of the nerves.

In another aspect of the present invention, a stimulation system may provide electric potential in order to stimulate nerves and/or to contract muscles of the standard pelvic structure of a female.

In one exemplary embodiment of this aspect of the invention, such a system may be made by a process including the steps of: arranging a first body member capable of receiving at least a portion of a penile structure of a male; arranging a second body member to receive at least a portion of such a pelvic structure; providing at least one power member capable of providing power for the potential; disposing at least one electrode member on at least one of the body members; arranging at least one sensor unit to measure at least one variable related to the system, the pelvic structure of the female, and/or a penile structure of a male; generating the potential by the power member when a value of the variable is arranged to exceed a preset value and/or to fall within a preset range; and delivering such potential to the pelvic structure by the electrode member as the first body member engages the pelvic structure, thereby causing contraction of the muscles and/or stimulation of the nerves.

In another exemplary embodiment of this aspect of the invention, such a system may be made by a process including the steps of: arranging a first body member to receive at least a portion of a penile structure of a male; arranging a second body member capable of receiving at least a portion of the pelvic structure; providing at least one power member which is capable of providing power for the potential; disposing at least one electrode member on at least one of the body members; arranging at least one sensor unit to measure at least one variable related to the system, the pelvic structure of the female, and/or a penile structure of a male; generating the potential by the power member in at least a substantial synchronization with the variable; and delivering the potential to the pelvic structure by the electrode member as the first body member engages the pelvic structure, thereby causing contraction of the muscles and/or stimulation of the nerves.

In another aspect of the present invention, a stimulation system may provide electric potential and then delivering the potential to muscles and/or nerves of the standard pelvic structure of a female, where such potential may be arranged to contract the muscles of the structure in a preset order.

In one exemplary embodiment of this aspect of the invention, such a system may be made by a process including the steps of: arranging a first body member to receive at least a portion of a penile structure of a male; providing at least one power member capable of providing power for the potential; disposing at least one electrode member on the first body member in an orientation matching that of the pelvic structure; generating the potential by the power member; and delivering the potential to the pelvic structure in a preset direction of the electrode member as the first body member engages the pelvic structure, thereby causing contraction of the muscles and/or stimulation of the nerves along an uniform direction. The delivering may be replaced by the step of: delivering the potential to the pelvic structure in alternating directions along the electrode member when the first body member engages the pelvic structure, thereby causing contraction of the muscles and/or stimulation of the nerves along opposing directions alternatingly.

In another exemplary embodiment of this aspect of the invention, such a system may be made by a process including the steps of: arranging a first body member to receive at least a portion of a penile structure of a male; providing at least one power member capable of providing power for such potential; disposing multiple electrode members on the first body member; generating such potential by the power member; and delivering the potential to the pelvic structure along each of multiple directions defined along the electrode member as the first body member engages the pelvic structure, thereby causing contraction of the muscles and/or stimulation of the nerves along each of multiple directions.

In another exemplary embodiment of this aspect of the invention, such a system may be made by a process including the steps of: arranging a first body member to receive at least a portion of a penile structure of a male; providing at least one power member capable of providing power for the potential; disposing multiple electrode members on such a first body member; arranging at least one sensor unit to measure at least one variable related to the system, the pelvic structure of the female, and/or a penile structure of a male; generating the potential by the power member; and delivering the potential to the pelvic structure in one of multiple directions based on the variable when the first body member engages the pelvic structure, thereby causing contraction of the muscles and/or stimulation of the nerves in a corresponding direction.

Further product-by-process claims may be constructed by modifying the foregoing preambles or their modifications of the above system and/or method claims and by appending thereto the above bodies or their modifications of the above system and/or method claims. Such process claims may be arranged to include one or more of the aforementioned features of the above system and/or method claims of the present invention.

As used herein, a term “pelvic structure” refers to an anatomic structure of sexual organs of a female. Such a “pelvic structure” typically defines an entry and a wall, where the entry is arranged to define an orifice therethrough, and where the wall is arranged to include various muscles and to also define an internal cavity which is arranged to extend inwardly and to be also bound by the muscles

As used herein, both of the terms “sensor unit” and “input unit” refer to those units of various pelvic quotient systems capable of receiving various variables of the pelvic structure (or user inputs) and/or monitoring dynamic pattern thereof. However, the “sensor unit” and the “input unit” are to be differentiated as follows within the scope of this invention. First, such a “sensor unit” is preferably disposed in an insertable part of a body member, while the “input unit” is generally disposed in a part of the body member of the system which is intended to not be inserted into the internal cavity of such a pelvic structure. Therefore, the “sensor unit” generally receives the user inputs (more specifically, various variables related to dynamic patterns and/or configurations of the pelvic structure or simply “pelvic variables”) through various portions of the pelvic structure, whereas the “input unit” primarily receives the user inputs through a hand or a finger of an user.

The terms “proximal” and “distal” will be used in a relative context. Throughout this invention, the term “proximal” is to be used to denote a direction toward a head of a body member of a system, whereas the term “distal” is to be used to represent an opposite direction toward an end of a handle of such a system. Accordingly, a “proximal” end and a “distal” end may be defined with respect to an entire pelvic quotient system or with respect to a specific member or unit thereof.

The terms “input unit” and “sensor unit” generally refer to identical or similar articles capable of monitoring various dynamic patterns of various user inputs applied thereto. Throughout this invention, however, the “input units” represent such articles incorporated into a handle part (or a second unit) of a body member of a pelvic quotient system, while the “sensor units” denote such articles incorporated into an insertable part (or a first unit) of such a body member. Accordingly, any articles which may be used as the “input unit” may also be used as the “sensor unit” unless otherwise specified. In addition, the “input unit” may also be disposed in the insertable part of the body member, while the “sensor unit” may also be disposed in the handle part thereof when desired.

As used herein, a “dynamic pattern” refers to a temporal pattern as well as a spatial pattern of a variable (i.e., a “pelvic variable”), of an user input, and/or of a sensing signal each of which may be generated by a sensor unit and/or an input unit of a control member in response to various variables (or user inputs), while a “dynamic feature” refers to a temporal feature as well as a spatial feature of various potentials generated by a power member and/or delivered along an electrode member. Thus, all definitions regarding the “dynamic pattern” may be applied to definitions of the “dynamic feature” as long as such definitions of the “dynamic feature” are described based upon the electric potentials.

Unless otherwise defined in the following specification, all technical and scientific terms used herein have the same meaning as commonly understood by one of ordinary skill in the art to which the present invention belongs. Although the methods or materials equivalent or similar to those described herein can be used in the practice or in the testing of the present invention, the suitable methods and materials are described below. All publications, patent applications, patents, and/or other references mentioned herein (particularly those enumerated in the above Background section) are incorporated by reference in their entirety. In case of any conflict, the present specification, including definitions, will control. In addition, the materials, methods, and examples are illustrative only and not intended to be limiting.

Other features and advantages of the present invention will be apparent from the following detailed description, and from the claims.

### BRIEF DESCRIPTION OF THE DRAWING

FIGS. 1A to 1D are schematic views of exemplary adaptive stimulation systems each of which is provided as a single-module system according to the present invention; and

FIGS. 2A to 2D are schematic views of exemplary adaptive stimulation systems each of which is provided as a dual-module system according to the present invention.

### DETAILED DESCRIPTION OF THE PREFERRED EMBODIMENTS

The present invention generally relates to systems capable of providing and delivering electric potential to a pelvic (i.e., vaginal) structure of a female to cause contraction of pelvic muscles and/or stimulation of pelvic nerves. More particularly, the present invention relates to various systems which include various power members and electrode members, where such power members are capable of providing electric power for such potential from various sources and where such electrode members are capable of delivering the potential along a preset direction or multiple directions. Therefore, such systems may also induce contraction of the pelvic muscles and/or stimulation of the pelvic nerves in a desirable order. Such systems may further induce the muscle contraction and/or nerve stimulation in synchronization with dynamic patterns of various variables of the system and/or the pelvic structure, where various sensor units of the systems may monitor movements of various members and/or units of the systems, dispositions thereof, and/or external perturbations and where control members of the systems may manipulate a timing of such potential, an amplitude thereof, and other dynamic features of such potential. Such systems may preferably obtain the power from various portable and compact sources such as, e.g., portable electromechanical generator capable of converting movement of the system and/or contact force applied thereto into electrical energy, portable piezoelectric generator for converting the contact force applied thereto into electrical energy, portable and mobile magnet and/or electric conductor moving across electric and/or magnetic fields and generating the potential through electromagnetic induction.

The present invention also relates to various methods of generating such electric potential and delivering such potential to the pelvic structure for inducing contraction of muscles and/or stimulation of nerves of the pelvic structure. More particularly, the present invention relates to various methods of providing various portable adaptive stimulation systems, generating power for such potential from various portable sources, delivering such potential by various electrode members, arranging multiple electrode members, and the like. The present invention also relates to various methods of measuring various system and/or structure variables and delivering such potential when values of the variables exceed a preset value (or fall within a preset range) and various methods of generating or delivering such potential to the pelvic structure in synchronization with such system and/or structure variables. The present invention also relates to various methods of synchronizing the muscle contraction and/or nerve stimulation with one or more variables of the system and/or structure, and various methods of inducing the muscle contraction and/or nerve stimulation in a preset order and/or pattern. The present invention further relates to various processes for providing various members, units, and/or parts of the system and, more particularly, various processes for making portable power members capable of supplying power for the potential, for incorporating the power members into wearable body members of the system, for incorporating various electrode members onto such body members, for providing control members capable of measuring various system and/or structure variables and synchronizing such potential with such variables, and the like.

Various aspects and/or embodiments of various systems, methods, and/or processes of this invention will now be described more particularly with reference to the accompanying drawings and text, where such aspects and/or embodiments thereof only represent different forms. Such systems, methods, and/or processes of this invention, however, may also be embodied in many other different forms and, accordingly, should not be limited to such aspects and/or embodiments which are set forth herein. Rather, various exemplary aspects and/or embodiments described herein are provided so that this disclosure will be thorough and complete, and fully convey the scope of the present invention to one of ordinary skill in the relevant art.

Unless otherwise specified, it is to be understood that various members, units, elements, and parts of various systems of the present invention are not typically drawn to scales and/or proportions for ease of illustration. It is also to be understood that such members, units, elements, and/or parts of various systems of this invention designated by the same numerals may typically represent the same, similar, and/or functionally equivalent members, units, elements, and/or parts thereof, respectively.

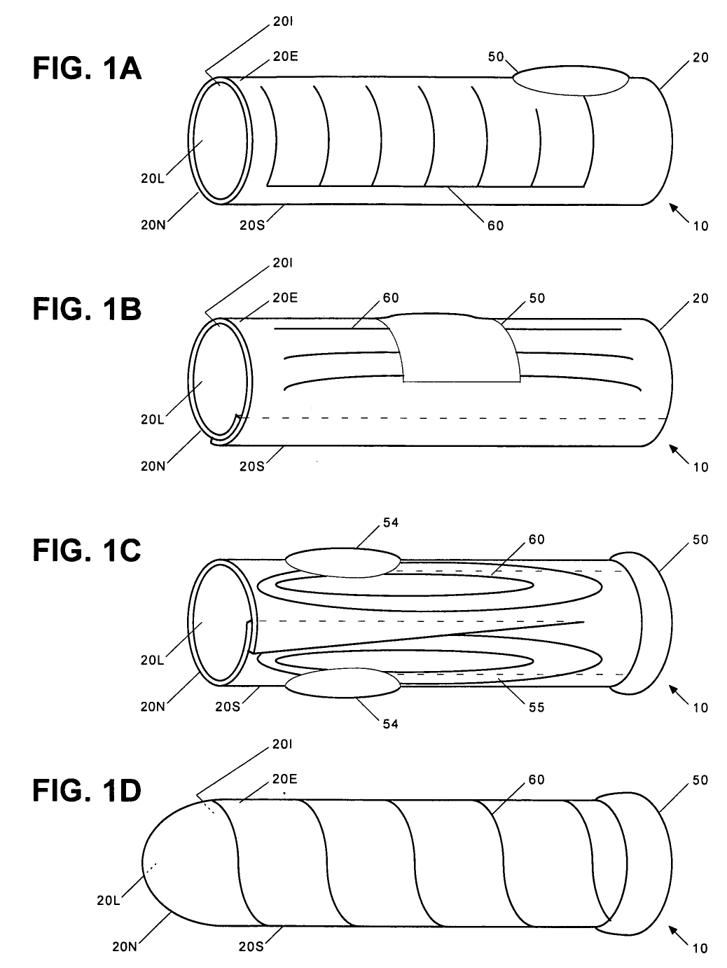

In one aspect of the present invention, such an adaptive stimulation system may be arranged to include at least one body member, at least one electrode member, and at least one power member all of which may be incorporated into a single-module system. FIGS. 1A to 1D are schematic views of various exemplary adaptive stimulation systems each of which is provided as a single-module system according to the present invention. It is appreciated in all of these figures that a direction to the left is a proximal direction and that an opposite direction is a distal direction. In other words, when such a system is put around a shaft of a penile structure of a male, a tip of the shaft is to be disposed on the left, while a base of the shaft is to be positioned on the right.

In one exemplary embodiment of such an aspect of the invention and as described in FIG. 1A, an exemplary adaptive stimulation system 10 includes a first body member 20, a power member 50, an electrode member 60, and the like. The first body member 20 is generally elongated, forms an annular cylinder, and defines a side 20S having an exterior surface 20E and an interior surface 20I. Bound by the interior surface 20I is a lumen 20L which also forms a shape of a cylinder. In this embodiment, the first body member 20 forms two opposing ends 20N both of which are open so that such a first body member 20 may receive a preset length of a shaft of a penile structure of a male. In addition, the first body member 20 also defines the same shape and size along its longitudinal axis such that a diameter of the lumen 20L on the proximal end is the same as that of the lumen 20L on the distal side. As will be described in greater detail below, however, such a first body member 20 is preferably made of or includes elastic materials such that its axial and radial dimensions may change in response to external force.

The power member 50 is disposed in a distal portion of the first body member 20. The power member 50 of this embodiment is a conventional battery storing therein electrical energy for generating electrical potential having an amplitude which is high enough to contract or “twitch” various muscles of a pelvic structure of a female and/or to stimulate various nerves of the pelvic structure. The power member 50 is preferably arranged to generate at least tens (or hundreds) of such potentials so as not to run out of such electrical energy during use. The battery-type power member 50 may be disposed on or over the exterior surface 20E of the first body member 20, on the interior surface 20I thereof, or inbetween. In addition, the power member 50 may also be disposed in a proximal or middle portion of the first body member 50.

The electrode member 60 is typically made of and/or includes electrically conductive materials and electrically connected to the power member 50. Accordingly, the electrode member 60 delivers the electric potential generated by the power member 50 therealong toward various parts of the first body member 20. In particular, a main stem of such an electrode member 50 extends along a preset length of the first body member 20 while branching off along a radial direction, where such branches of this embodiment are disposed in at least substantially identical intervals. Therefore, the electrode member 60 defines a comb-shaped network which covers a substantial part of the exterior surface 20E of the first body member 20. It is appreciated that the electrode member 60 of this embodiment forms an open circuit, i.e., the electrode member 60 does not include a return path back to the power member 50. Accordingly, various branches of the electrode member 60 may not have to couple with each other.

Although not shown in the figure, the system 10 may include a control member which may be arranged to control various operations of the system 10. A major function of the control member may be to control timing of the electric potential, i.e., a firing sequence of such electric potentials along the electrode member 60. As will be described in greater detail below, the control member may determine such timing based on various control algorithms.

In operation, the shaft of the penile structure is inserted through the lumen 20L of the first body member 20 into a preset depth, while exposing a tip of the penile structure through one end 20E of the body member 20. As a result, a major portion of the shaft of the penile structure is to be enclosed or covered by the side 20S of the first body member 20 and the tip of the penile shaft is exposed through the open proximal end 20N of the first body member 20. Thereafter, the male inserts the shaft of his penile structure into the internal cavity of the pelvic structure of his partner through the pelvic orifice and commences reciprocating movements. Along therewith, the first body member 20 is disposed in the internal cavity as well and the electrode member 60 is brought into contact with the pelvic wall. Based on a timing control (or firing sequence) algorithm, the control member draws a preset amount of energy from the power member 50, generates the electric potential, and then releases such potential therefrom. Such potential is then transmitted through the electrode member 60 to various portions of the pelvic structure. Because the pelvic wall itself is an electric conductor and also secrets aqueous conductive fluids during sexual intercourse, such potential is delivered from the electrode member 60 to various portions of the pelvic structure, thereby causing contraction of the pelvic muscles and/or stimulation of the pelvic nerves. It is appreciated that numerous branches of the electrode member 60 extends radially or circumferentially along the same direction. Because the electric potential may then be delivered through such branches along the same direction, it is feasible that the pelvic muscles and nerves may be respectively contracted and stimulated systematically.

In another exemplary embodiment of such an aspect of the invention and as described in FIG. 1B, another exemplary adaptive stimulation system 10 also includes a first body member 20, a power member 50, and an electrode member 60. Such a first body member 20 is a generally elongated sheet having an exterior (or upper) surface 20E and an interior (or lower) surface. Opposing edges of the first body member 20 are preferably arranged to be releasably coupled to each other when disposed one over the other. In particular, one edge of the interior surface 20I includes a conventional coupling mechanism for releasably coupling with opposing edge of the exterior surface 20E, where examples of the coupling mechanisms may include, but not be limited to, adhesives, Velcro's, ridges, and so on. Accordingly, the first body member 20 also defines a longitudinal axis and forms a lumen 20L therein, and also defines two opposing open ends 20N when rolled or wrapped therearound and coupled to each other along such edges. The first body member 20 of this embodiment defines the same shape and size along its length such that a diameter of the lumen 20L on the proximal end is the same as that of the lumen 20L on the distal side. Thus, the first body member 20 forms a seam (represented by a dotted line) extending between the opposing ends 20N. The first body member 20 is preferably made of and/or includes elastic materials so that its axial and radial dimensions may change in response to external force.

The power member 50 is disposed in a middle portion of the first body member 20. The power member 50 of this figure is an electromechanical generator capable of converting various mechanical energy applied thereto into electrical energy and then generating electrical potential having the similar amplitude therefrom. One example of this power member 50 is a conventional piezoelectric material or article capable of converting force, momentum or energy applied thereto into the electrical energy. In general, such piezoelectric material does not undergo a noticeable deformation while generating such electrical energy. Another example of the power member 50 is an electromechanical assembly having a movable unit and a magnet (neither shown in this figure), where the movable unit is made of and/or includes an electric conductor. The power member 50 is arranged such that, upon receiving external force or energy or abutting the pelvic structure, the movable unit moves across a magnetic field which is generated by the magnet, and then generates an electric current through electromagnetic induction. Another example of the power member 50 is a thermal generator which generates the electric current and voltage when subjected beyond a certain temperature. This power member 50 typically includes one or more chemicals which undergo a phase transition and/or chemical reaction beyond the preset temperature. The power members 50 of such first two examples may generate the electric potential whenever they receive the external force or energy and/or whenever they are subjected to a motion. Accordingly, such power members 50 may be used for an indefinite duration. The power member 50 of the last example, however, lasts for a certain period of time until such chemicals are all consumed. Accordingly, such a power member 50 is preferably arranged to generate at least tens (or hundreds) of such potentials so as not to run out of such electrical energy during use. Such power members 50 may also be disposed on or over the exterior surface 20E of the first body member 20, on the interior surface 20I thereof, or inbetween. The power member 50 may also be disposed in a proximal and/or distal portion of the first body member 50.

The electrode member 60 is also electrically conductive and connected to the power member 50 in order to deliver such electric potential generated by the power member 50 therealong to various parts of the first body member 20. Contrary to that of FIG. 1A, the electrode member 60 of this figure includes multiple conductive stems extending somewhat parallel to each other along the longitudinal direction. Therefore, multiple stems of such an electrode member 60 may cover a substantial part of the exterior surface 20E of the first body member 20. It is appreciated that the electrode member 60 of this embodiment also forms an open circuit without any return path back to the power member 50.

The system 10 may also include a control member which may be arranged to control various operations of the system 10. A major function of the control member may be to control timing of such electric potential, i.e., a firing sequence of such electric potentials along the electrode member 60. As will be described in greater detail below, the control member may determine such timing according to various control algorithms.

In operation, the sheet-like first body member 20 is placed along a desirable length of the shaft of the penile structure, with its coupling edges aligned parallel to the shaft. One coupling edge is then wrapped around the shaft, placed over the exterior surface 20E of the opposing edge, and releasably coupled or fastened thereto by applying pressure thereon. In this state, the tip of the penile structure is exposed through the proximal end of the first body member 20, while a major portion of the shaft of the penile structure is enclosed or covered by the side 20S of the first body member 20. Thereafter, the male inserts the shaft of his penile structure into the internal cavity of the pelvic structure of his partner through the pelvic orifice and commences reciprocating movements. Along therewith, the first body member 20 is disposed in the internal cavity as well and the electrode member 60 is brought into contact with the pelvic wall. The reciprocating movements of the first body member 20 put the power member 50 in motion, from which the power member 50 receives the external force or energy exerted by the pelvic structure and then converts at least a portion of such force or energy into the electrical energy. Alternatively, the movable unit may move with respect to the magnet, and the power member 50 may generate the electrical energy. In another alternative, the power member 50 may generate the electrical energy through the phase change and/or reaction of the chemicals contained therein or may generate such energy by utilizing the thermal energy from the pelvic structure. Regardless of detailed mechanisms of the power member 50, the control member releases the electric potential generated by such a power member 50 to the electrode member 60 which then distributes the potential onto various portions of the pelvic structure. Because the pelvic wall itself is an electric conductor and secrets the aqueous conductive fluids during sexual intercourse, such potential is delivered to various portions of the pelvic structure, thereby causing contraction of the pelvic muscles and/or stimulation of the pelvic nerves. It is to be understood that multiple stems of the electrode member 60 extends along the axial or longitudinal direction. Because the electric potential may be delivered through such stems along the same direction, it is feasible that such pelvic muscles and nerves may be respectively contracted and stimulated systematically. Other configurational and/or operational characteristics of the system 10 of FIG. 1B are similar or identical to those of the system of FIG. 1A.

In another exemplary embodiment of such an aspect of the invention and as described in FIG. 1C, another exemplary adaptive stimulation system 10 also includes a first body member 20, a power member 50, and an electrode member 60. The first body member 20 is generally elongated, forms an annular cylinder, and includes a side 20S having an exterior surface 20E and an interior surface 20I. Bound by the interior surface 20I is a lumen 20L having a shape of a cylinder. In this embodiment, the first body member 20 forms two opposing ends 20N both of which are open so that such a first body member 20 may receive a preset length of a shaft of a penile structure of a male. It is appreciated that the proximal end 20N of such a body member 20 is arranged to open on one side in order to facilitate position of the shaft of the penile structure therethrough. Therefore, opposing edges of the open part of such a member 20 are preferably arranged to be releasably coupled to each other when disposed one over the other through conventional coupling mechanisms as described in conjunction with FIG. 1A. The first body member 20 of this embodiment defines the same shape and size along its length so that a diameter of the lumen 20L on the proximal end is approximately the same as that of the lumen 20L on the distal side. Such a body member 20 is preferably made of and/or includes elastic materials so that its axial and radial dimensions may change in response to external force.

The power member 50 includes a main unit disposed in the distal end of the first body member 20 and multiple movable units 54 each of which is mechanically and/or electrically coupled to the main unit through a coupling unit 55. The power member 50 of this figure is an electromechanical generator which is similar to that of FIG. 1B, therefore, converts various mechanical energy applied thereto into electrical energy, and generates the potential. The power member 50 incorporating such multiple units may offer various advantages. In one example, such movable units 54 may be disposed in locations in which the first body unit 20 tends to receive the most external force or energy, thereby maximizing an amplitude of the potential. In another example, various parts of such a power member 50 may be distributed in multiple units in various embodiments which may facilitate positioning of such a first body member 20. Accordingly, various stationary units of the power member 50 may be disposed in such a location to which the least external force or energy is applied. In the embodiment of FIG. 1C, the main unit is shaped as an annular ring and disposed around a periphery of the distal end of the first body member 20, and the movable units 54 are disposed at a distance from the main unit. More particularly, the movable unit 54 may be arranged to change its configuration and/or disposition in response to the external force or energy, while the change is then transmitted to the main unit through a conventional power transmission article to the main unit, in which the electrical energy may be generated by one or more of the aforementioned mechanisms. Such power members 50 may be disposed on or over the exterior surface 20E of such a body member 20, on its interior surface 20I thereof, or inbetween. The power member 50 may be disposed in a proximal and/or distal portion of the first body member 50.

The electrode member 60 is also electrically conductive and connected to the power member 50 in order to deliver such electric potential generated by the power member 50 therealong to various parts of the first body member 20. Contrary to those of FIGS. 1A and 1B, the electrode member 60 of this figure includes multiple conductive loops defining multiple concentric configurations and originating from each movable unit 54 of the power member 50. Accordingly, the electric potential generated by each movable unit 54 (or such potential generated by the main module of the power member 50 and delivered to each movable unit 54) may be distributed to the concentric loops of each movable unit 54. Therefore, multiple loops of such an electrode member 60 may cover a substantial part of the exterior surface 20E of the first body member 20.

The system 10 may also include a control member which may be arranged to control various operations of the system 10. A major function of the control member may be to control timing of such electric potential, i.e., a firing sequence of such electric potentials along the electrode member 60. As will be described in greater detail below, the control member may determine such timing according to various control algorithms.

In operation, the shaft of the penile structure is inserted through the lumen 20L of the first body member 20 into a preset depth, while exposing a tip of the penile structure through one end 20E of the body member 20. One coupling edge of the proximal end 20N of the first body member 20 is wrapped around the shaft, placed over the exterior surface 20E of a corresponding edge, and then releasably coupled or fastened thereto by applying pressure thereon. As a result, the tip of the penile structure is exposed through the proximal end of the first body member 20, while a major portion of the shaft of the penile structure is enclosed or covered by the side 20S of the first body member 20. Thereafter, the male inserts the shaft of his penile structure into the internal cavity of the pelvic structure of his partner through the pelvic orifice and commences reciprocating movements. Along therewith, the first body member 20 is disposed in the internal cavity and the electrode member 60 is brought into contact with the pelvic wall. Such reciprocating movements of the first body member 20 put the movable unit 54 of the power member 50 in motion, from which the power member 50 receives the external force or energy exerted by the pelvic structure and then converts at least a portion of such force or energy into the electrical energy. Alternatively, the movable unit may move with respect to the magnet, and the power member 50 may generate the electrical energy. In another alternative, the power member 50 may generate the electrical energy by the phase change and/or reaction of the chemicals included therein or may generate the energy by using the thermal energy from the pelvic structure. Regardless of detailed mechanisms of the power member 50, the control member releases such electric potential generated by the power member 50 to the electrode member 60 which then distributes such potential onto various portions of the pelvic structure. Because the pelvic wall itself is an electric conductor and secrets the aqueous conductive fluids during sexual intercourse, such potential may be delivered to various portions of the pelvic structure, thereby causing contraction of such pelvic muscles and/or stimulation of the pelvic nerves. It is to be understood that each group of multiple concentric loops of the electrode member 60 may enclose only a part of the first body member 20 and, therefore, that the potential generated by such a group may be confined to only a portion of the pelvic structure. Thus, the power member 50 of this embodiment may deliver the potential and cause such contraction and/or stimulation locally. By the same token, such a power member 50 may also deliver to different portions of the pelvic structure such potentials defining different amplitudes and/or other dynamic patterns as will be provided in greater detail below. Other configurational and/or operational characteristics of the system 10 of FIG. 1C are similar or identical to those of the systems of FIGS. 1A and 1B.

In another exemplary embodiment of such an aspect of the invention and as described in FIG. 1D, another exemplary adaptive stimulation system 10 also includes a first body member 20, a power member 50, and an electrode member 60. The first body member 20 is generally elongated, forms an annular cylinder which is typically similar to those of FIGS. 1A to 1C. In this embodiment, however, the first body member 20 forms an open distal end 20N but a closed proximal end 20N, similar to a shape of a conventional condom. The first body member 20 of this embodiment defines the same shape and size along its length so that a diameter of the lumen 20L on the proximal end is approximately the same as that of the lumen 20L on the distal side. Such a body member 20 is also preferably made of and/or includes elastic materials so that its axial and radial dimensions vary in response to external force.

The power member 50 is disposed in a distal portion of the first body member 20. The power member 50 of this figure is an electromechanical generator which may be similar to that of FIG. 1B so that such a member 50 may convert various mechanical energy applied thereto into electrical energy and generate electrical potential having the similar amplitude therefrom. Therefore, the power member 50 may be any of the aforementioned conventional piezoelectric material, electromechanical assembly, thermal generator, and so on. Such power members 50 may also be disposed on or over the exterior surface 20E of such a body member 20, on its interior surface 20I, inbetween, and so on. The power member 50 may also be disposed in a proximal and/or distal portion of the first body member 50.

The electrode member 60 is also electrically conductive and connected to the power member 50 in order to deliver such electric potential generated by the power member 50 therealong to various parts of the first body member 20. Contrary to those of FIGS. 1A to 1C, such an electrode member 60 of this figure includes a single conductive loop helically wound around the exterior surface 20E of the first body member 20. Therefore, the helical loop of the electrode member 60 may cover a substantial part of the exterior surface 20E of the first body member 20. When desirable, the electrode member 60 may include multiple helical loops would in the same of different directions, wound in the same or different pitches, and the like. It is appreciated that the electrode member 60 of this embodiment forms an open circuit without any return path back to the power member 50.

The system 10 may also include a control member which may be arranged to control various operations of the system 10. A major function of the control member may be to control timing of such electric potential, i.e., a firing sequence of such electric potentials along the electrode member 60. As will be described in greater detail below, the control member may determine such timing according to various control algorithms.

In operation, the shaft of the penile structure is inserted through the lumen 20L of the first body member 20 into a preset depth, while enclosing a tip of the penile structure inside the proximal end 20E of the body member 20. As a result, a major portion of the shaft of the penile structure including its tip is enclosed and/or covered by the first body member 20. The male then inserts the shaft of his penile structure into the internal cavity of her pelvic structure through the orifice of the structure, and starts reciprocating movements. Along therewith, the first body member 20 is disposed in the internal cavity and the electrode member 60 contacts the pelvic wall. The reciprocating movements of the first body member 20 put the power member 50 in motion, and the power member 50 receives the external force or energy exerted by the pelvic structure and then converts at least a portion of such force or energy into the electrical energy. Alternatively, the power member 50 may have the movable unit moving with respect to its magnet, and the power member 50 generates such electrical energy. In the alternative, the power member 50 may generate the electrical energy by the phase change and/or reaction of the chemicals included therein or generate the energy by using the thermal energy of the pelvic structure. The control member releases such potential generated by the power member 50 toward the electrode member 60 which distributes such potential onto various portions of the pelvic structure. Because the pelvic wall itself is an electric conductor and also secrets the aqueous conductive fluids during sexual intercourse, the potential may be delivered to various portions of the pelvic structure, thereby causing contraction of such pelvic muscles and/or stimulation of the pelvic nerves. It is to be understood that the helical loop of the electrode member 60 may be shaped and/or sized to match another helical loop of the muscles of the pelvic structure. Thereby, the potential generated by the power member 50 of this embodiment may deliver the potential and cause such contraction and/or stimulation locally in an orderly pattern. Other configurational and/or operational characteristics of the system 10 of FIG. 1D are similar or identical to those of the systems of FIGS. 1A to 1C.

Configurational and/or operational variations and/or modifications of the above embodiments of the exemplary systems and various members thereof described in FIGS. 1A through 1D also fall within the scope of this invention.

The above first body member may define any shapes and/or sizes, with an only restriction that such a member may be able to receive thereinto and then to retain therein at least a (major) portion of the shaft of the penile structure. As mentioned above, the first body member may be made of and/or include the elastic materials such as thermoplastic polymers. In this case, the first body member may define such a shape and/or size as the member is stretched to its stressed position. It is particularly important that the first body member retains the penile shaft thereonto during use in order to prevent the body member to be stripped thereof by vigorous movements accompanying a sexual intercourse. The first body member may be provided as an annular cylinder with two open ends as exemplified in FIGS. 1A to 1C or, in the alternative, as another annular cylinder with one closed end as exemplified in FIG. 1D. Accordingly, the system defining the latter embodiment may also be used as a contraceptive device.

The main function of the first body member is to house and/or to support the electrode member disposed thereon. It therefore follows that the first body member is preferably disposed to contact a preset portion(s) of the pelvic structure when the penile structure onto which the system is disposed engages the pelvic structure and that such a body member exposes at least a substantial part of the electrode member, thereby allowing such electric potential to be delivered to such a portion(s) of the pelvic structure through the electrode member. Thus, the electrode member may have to be disposed into or onto the first body member accordingly when the first body member may define a deformable configuration.

The first body member is generally disposed around the shaft of the penile structure, e.g., by wearing such a body member on the such a shaft, by inserting the shaft through the body member, by wrapping the body member around the shaft, and the like. In order to facilitate such dispositions, the first body member may be provided to be elastic, may define coupling edges as exemplified in FIG. 1B, may include a partially open end as shown in FIG. 1C, and the like. When such a body member relies on coupling its edges for proper disposition, care should be taken for avoiding the body member from being stripped off during use and for not creating any sharp edges along the coupling edges.

In general, the first body member may be made of and/or include any materials as long as such a body member may properly receive thereinto and retain therein a desired portion of the shaft of the penile structure. In one example, the first body member may be made of and/or include conventional thermoplastics defining suitable elasticity but durability. An adjustable configuration obtained thereby may prove beneficial for sanitary, storage, use, and/or contraception purposes. It is appreciated that the first body member may be provided as a disposable article. To this end, such a member may also be made of and/or include relatively economical materials. When desirable, such a member may also be provided as an reusable article, which may accompany proper washing and sanitizing after each use. In this case, such a first body member may preferably be made of and/or include more durable materials for repeated use. In addition, at least a part of the first body member may be made of and/or include rigid materials for various reasons such as, e.g., maintaining its configuration, resisting wear and tear during use, and so on. In general, the first body member is made of and/or include electrically insulative materials so that the electric potential delivered to the pelvic structure through the electrode member disposed on its exterior surface may not be transmitted onto the interior surface and contract the penile muscles and/or stimulate penile nerves. To the contrary, the first body member may instead be made of and/or include electrically conductive materials when it is desirable to contract the penile muscles and/or to stimulate the penile nerves.

The first body member may define a single-layered article which may be homogeneous or, in the alternative, may include multiple inhomogeneous regions, where some of such regions may define mechanical, electrical, and/or chemical properties different from those of at least one of the rest of the regions. Such a first body member may also form an article with multiple layers, where some of such layers may have mechanical, electrical, and/or chemical properties different from those of at least one of the rest of the layers. Accordingly, the first body member may define an articulate article defining a preset conductive path therealong and/or thereacross.

The above electrode member may define any shapes and/or sizes, with an only restriction that the electrode member may be able to deliver the electric potential onto a preset portion(s) of the pelvic structure. It therefore follows that the electrode member is electrically coupled to the power member to receive the potential either through wire or wirelessly and that the electrode member is distributed on, over or across the first body member in a pattern ensuring such potential to be delivered to such a portion(s) of the pelvic structure.

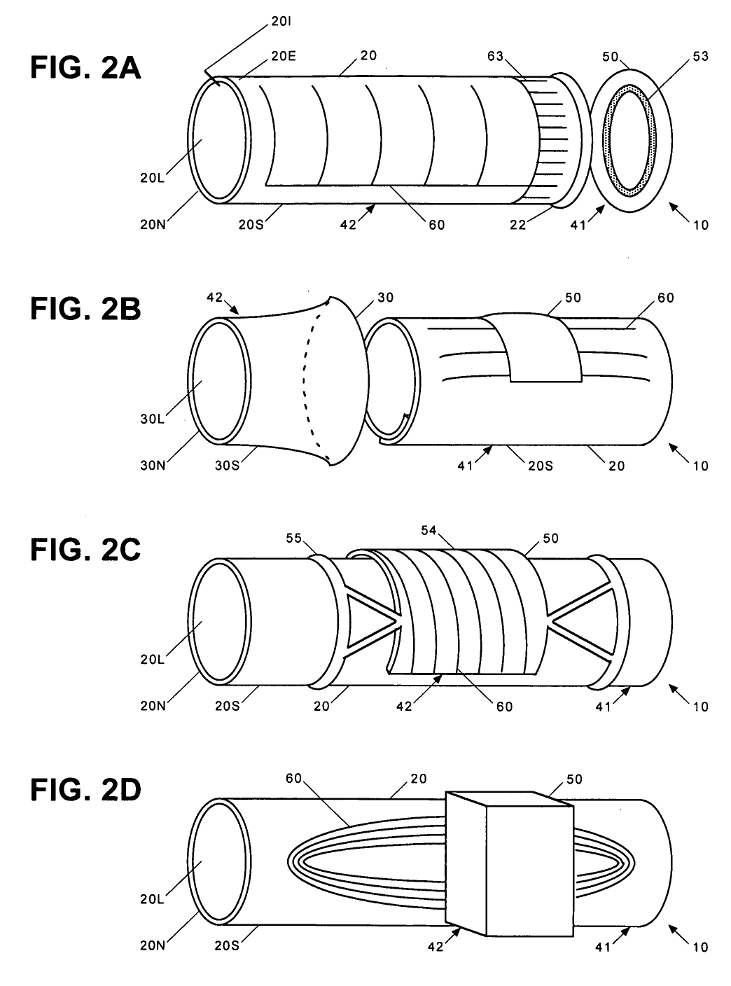

As described above, the electrode member or at least a substantial part thereof is preferably disposed on or over the first body member and supported thereby. Depending upon configuration of the system, such an electrode member may be disposed adjacent to or away from the power and/or control members. In some cases, such an electrode member may instead be disposed on or over the power and/or control members which may be disposed on or in the first body member or may instead be disposed farther away from or detached from such a first body member. In addition, the electrode member may be disposed in various arrangements with respect to other members when the system consists of multiple modules as will be described in greater detail in conjunction with FIGS. 2A to 2D.

The electrode member may also include one or multiple stems, loops, and/or other branches as briefly exemplified in FIGS. 1A to 1D. It is appreciated, however, that detailed configuration of such an electrode member may be determined based upon numerous design factors such as, e.g., portions of the pelvic structure to which the potential is to be delivered, locations of such portions, area of such portions, orientation of the pelvic muscles and/or nerves in such portions, configuration of the pelvic structure adjacent to such portions, configuration of the first body member, a location of such a body member onto which the electrode member is to be incorporated, an area of such a location, and so on. Accordingly, the electrode member may be designed as a single-spot or multiple-spot electrode, as an area electrode, and the like, where shapes and sizes of each spot and area thereof may be decided based upon the above design factors. It is appreciated that such an area electrode may be arranged to extend along the axial, circumferential or radial direction along or around the first body member. In the alternative, the area electrode may also wind the first body member in a helical pattern. It is also appreciated that the electrode member may include multiple spot electrodes which may be arranged to simulate the above area electrodes by distributing such spot electrodes in similar arrangements. As far as such an electrode member may deliver the electric potential to the desired portion of the pelvic structure in a desirable order, the detailed configuration of such electrodes may not be material to the scope of the present invention.

As described above, the electrode member may be arranged to excite the pelvic muscles and nerves in order to achieve ordered contraction of such muscles and/or stimulation of such nerves. In one example, the electrode member may be arranged to induce such contraction and/or stimulation in an uniform or constant direction along a preset path of conduction. In another example, the electrode member may be arranged to induce such contraction and/or stimulation in alternating directions along a conduction path. In yet another example, the electrode member may instead be arranged to induce the contraction and/or stimulation in varying directions along each of multiple conduction paths whether in a preset order or randomly. In another example, the electrode member may be arranged to induce the contraction and/or stimulation along one or multiple directions which may be decided by such dynamic patterns of system, pelvic, and/or penile variables and/or by the dynamic features of the potential. In all of these example, the electrode member may include one or multiple point electrodes and/or one or multiple area electrodes and direct the electric potential accordingly. It is appreciated that the detailed arrangements of such electrodes and/or firing sequence thereof may preferable be based upon the pelvic anatomy, in particular, orientation of the pelvic muscles defining exterior surfaces of the internal cavity of such a structure. In addition, the above electrode member and their electrodes may also be used to obtain ordered contraction of penile muscles and/or stimulation of penile nerves in an ordered pattern as long as details of the penile anatomy may be accounted for.

In addition to the above provisions, such electrode potential may be delivered thereto based on various timing schemes. For example, such potential may be delivered to the electrode member and/or their electrodes without any timing difference therealong. In such a case, the pelvic muscles and/or nerves may be excited almost simultaneously. In another example, the control member may deliver the potential to multiple electrodes with a time lag or in different timings so that the muscles and/or nerves may be excited in a timed pattern. Such an embodiment is generally preferred in attaining systematic contraction of the pelvic muscles and/or stimulation of the pelvic nerves. In another example, one or multiple capacitors or other electronic elements may be disposed along the conduction path such that the electric potential may travel along the conduction path with a preset timing difference. In another example, lengths of the electrode may be manipulated in order to obtain the desired firing sequence of multiple electrodes. In another example, the control member may be arranged to adaptively control the firing sequence of multiple electrodes depending upon one or more variables of the system, the pelvic structure, and/or the penile structure. When desirable, the control member may also allow the user to adjust the firing sequence or other operational details of delivering the potential to various electrodes of the electrode member.

Multiple electrode members and/or their electrodes may also be electrically arranged in various embodiments. In one example, such members and/or electrodes may be individually connected to the power member and. therefore, receive the same potential or different potentials therefrom. In another example, such members and/or electrodes may be connected in a parallel, series or hybrid pattern so that the potential may be distributed along the network of such electrode members and/or electrodes. When desirable, the electrode member may also include connection wire so as to deliver the potential from the power member thereto, from one location to another along the electrode member, and so on. Alternatively, such potential may be transmitted wirelessly from the power member and/or an external device as will be described below. In another alternative, the electrode member may generate such potential through electromagnetic induction. In this case, the electrode member may be deemed to use energy of an internal or external magnetic field which may then serve as a power member. In another alternative, the electrode member may be arranged to be at least partially magnetic and to generate the potential by utilizing energy of an internal or external electric field which may in turn serve as a power member.

The electrode member may generally be made of and/or include any conventional conductive materials defining a wide range of electric conductivity (or resistivity). Considering that the muscles may elicit contraction by an electric potential with an amplitude typically in the range of microvolts, the electrode member may be conductive or even semiconductive when the power member may generate a relatively strong evoking voltage. When the power member may be able to only generate a relatively weak evoking voltage, however, the electrode member may preferably be fairly conductive in order to minimize voltage drop therealong. Therefore, such an electrode member may be made of conventional metals or semiconductors a thin strip of which may be glued or otherwise attached on the exterior (or interior) surface of such a first body member. The conductive metals or semiconductors may instead be deposited and/or impregnated onto such a surface of the first body member by various mechanical or chemical processes commonly employed in the semiconductor industry. At least a part of the first body member may be similarly doped to an electric conductor as well. Such an electrode member may be of conventional conductive polymers a thin strip of which may be glued or otherwise attached onto the first body member. In the alternative, the conductive polymer may be included in a desirable part of the first body member during a manufacturing process or, alternatively, such a part of the first body member may be either chemically or electrically treated into a conductive polymer during and/or after the manufacture.

In general, the electrode member may be made of and/or include any materials as long as such an electrode member may deliver the electric potential therealong. In one example, such an electrode member may be made of and/or include conventional conductive thermoplastics which define suitable conductivity and elasticity but durability. This adjustable configuration may be beneficial for sanitary, storage, use, and/or anticorrosion purposes. It is appreciated that such an electrode member may be provided as a disposable article. To this end, the electrode member may be made of and/or include relatively economical materials. When desirable, such a member may also be provided as an reusable article, which may accompany proper washing and/or sanitizing after each use. In such a case, the electrode member may preferably be made of and/or include more durable materials for repeated use. In addition, at least a part of the electrode member may be made of and/or include rigid materials for various reasons such as, e.g., maintaining its configuration, resisting wear and tear during use, and so on. In general, various mechanical properties of the electrode member may be determined hand in hand with those of the first body member.

The electrode member may define a single-layered article which may be homogeneous or, in the alternative, may include multiple inhomogeneous regions, where some of such regions may define mechanical, electrical, and/or chemical properties different from those of at least one of the rest of the regions. Such an electrode member may also form an article with multiple layers, where some of the layers may have mechanical, electrical, and/or chemical properties different from those of at least one of the rest of the layers. Accordingly, the electrode member may define an articulate article defining a preset conductive path therealong and/or thereacross.

The above power member may define any shapes and/or sizes, with a sole restriction that the power member may be able to generate the electric potential which has the magnitude high enough to contract the pelvic (and/or penile) muscles and/or to stimulate the pelvic (and/or penile) nerves when the system engages with the pelvic (and/or penile) structure. It therefore follows that such a power member may be portable as well as compact enough to be incorporated into the system and that such a system may be fairly efficient enough to generate the potential with the above magnitude.

The power member may generate the electrical energy from various sources. In one example and as exemplified in FIG. 1A, a conventional battery may be used as the power member, where such a battery may be a dry-cell battery, a rechargeable battery, other compact or miniature batteries, and the like. Such a battery may store the electrical energy in an amount only for a single use (which may correspond to the amount to produce tens or hundreds of potentials) and, therefore, may be used as a disposable article. Alternatively, the battery mat store the electrical energy in another amount which may last multiple uses and, when desirable, may be recharged. Because such a battery may be able to supply the energy regardless of its position, such a power member may be disposed in almost any locations on, over, inside, and/or under the first body member.

In another example and as exemplified in FIG. 1B, such a power member may instead include an electromechanical generator capable of converting the contact force, momentum or energy into the electrical energy. Such a generator may typically include a transducer which is capable of receiving and converting such external perturbation into the electrical energy while undergoing deformation or without any deformation. Any conventional materials and/or assemblies capable of performing such conversion may be utilized as such a power member. Because the power member of this type needs to receive the external perturbation, it is necessary that such a power member be disposed over or in such locations which ensure such mechanical contact between at least a part of the power member and at least a portion of the pelvic (and/or penile) structure. Accordingly, the power member may be disposed on, in, under or along the body member which may contact the tip, shaft, and/or base of the penile structure, which may abut the internal cavity and/or orifice of the pelvic structure, and the like. When desirable, the power member may be incorporated as a member of the system but away from the body member. For example, the power member may be arranged to be actuated (i.e., receiving the external perturbation) by various body portions of the user except the pelvic (or penile) structure. In such a case, the power member may convert the contact force, momentum or energy applied by a finger, a hand, an arm, a shoulder, a tow, a leg, a thigh, a back, an abdomen, an upper torso, a head, and the like. The electrical energy generated thereby may then be transmitted to the electrode member through wire or wirelessly. When the system includes multiple modules as will be described below, the power member may be incorporated into a module which may not include the body member. In the alternative, the power for the potential may be supplied from external sources such as, e.g., external audiovisual devices, external communication devices, internet, and the like. Such energy may also be transmitted to the electrode member through wire or wirelessly.

In another example and as shown in FIGS. 1C and 1D, the power member may include another electromechanical generator capable of converting movement of at least a part of the system into the electrical energy. Such an electromechanical generator may include at least one movable unit and at least one stationary unit, where such a movable unit may translate, rotate or otherwise move relative to the stationary unit in order to generate such electrical energy by electromagnetic induction. Thus, when the movable unit includes a permanent magnet and/or an electromagnet, the stationary member includes an electric conductor. Conversely, when the movable unit is the electric conductor, then the stationary unit is the permanent magnet or electromagnet. It is appreciated that other configurations may also be used as long as the movable and stationary units cooperate to convert the movement of the movable unit into the electrical energy by electromagnetic induction. In addition, any conventional materials and/or assemblies capable of performing such conversion may be utilized as such a power member. Because the power member of this type needs to move with the system, it is necessary that such a power member be disposed over or in such locations which ensure its movement along with movement of the pelvic (and/or penile) structure. Therefore, the power member may be disposed on, in, under or along the body member which may move during use. When desirable, the power member may be incorporated as a member of the system but away from the body member. For example, the power member may be arranged to be moved by various body portions of the user except the pelvic (or penile) structure. In this case, such a power member may then be moved by the finger, hand, arm, shoulder, tow, leg, thigh, back, abdomen, upper torso, head, and the like. The electrical energy from such movement may then be transmitted to the electrode member through wire or wirelessly. When the system includes multiple modules, the power member may be included in a module which may not include the body member. In the alternative, the power for the potential may be supplied from external sources such as, e.g., external audiovisual devices, external communication devices, internet, and the like. Such energy may also be transmitted to the electrode member through wire or wirelessly. It is to be understood that the movable and/or stationary units of this type of power member may be provided as an external unit of the system or as an unit which may not belong to the system. For example, the stationary unit may be disposed external to the system and generate the external electric or magnetic fields in which the system may move during use, thereby allowing the movable unit of the system to generate the electrical energy.

Such a power member may convert the electrical energy stored and/or generated thereby into the electric potential and then deliver such potential to the electrode member based upon various firing schemes. In one example regarding the electromechanical generators, the power member may deliver the potential upon generating and converting the electrical energy thereinto. Such an embodiment may not be beneficial, for such potential may be premature (i.e., lacking in its magnitude) when the power member may not receive sufficient external perturbation and/or may not generate sufficient movement of the movable part. Accordingly and in another example, the power member may deliver the potential only if the amplitude of such potential exceeds a preset value. Although this power member ensures every released potential to evoke the desired excitation of the muscles and/or nerves, such a power member requires at least one storage unit capable of storing weak electrical energy until a sufficient amount thereof may be accumulated therein. In another example, the power member may be arranged to store the electrical energy, to convert such into the potential, and to release the potential thereafter. In another example, the power member may optionally include such a storage unit in order to store the electrical energy and release the potential thereafter upon user's command, regardless of whether an amplitude of the electrical energy generated by a single contact or movement may or may not exceed the preset value. In another example, the power member may further be arranged to convert a certain amount of the electrical energy into the potential, to manipulate the amplitude of the potential, to deliver such potential in a certain timing, and the like, where such an amount, amplitude, and/or timing may be determined by the movement of the body member and/or user, contact with the pelvic (and/or penile) structure, disposition of the body member, and/or other variables of the system, pelvic structure, and penile structure. In all of these examples, the control member may also be arranged to allow the user to manipulate the amplitude, timing, and/or other dynamic features of each potential.

In another aspect of the present invention, an adaptive stimulation system may also include at least one body member, at least one electrode member, and at least one power member, where each member may be incorporated into one or multiple modules of a multiple-module system. FIGS. 2A to 2D are schematic views of exemplary adaptive stimulation systems each of which is provided as a dual-module system according to the present invention. It is to be understood that the multiple-module systems of this aspect of the invention are generally similar to the single-module systems of FIGS. 1A to 1D, except that the multiple-module systems may provide more versatility in designing and utilizing such systems.

In one exemplary embodiment of such an aspect of the invention and as described in FIG. 2A, an exemplary adaptive stimulation system 10 may include a first module 41 and a second module 42, where the first module 41 includes a power member 50, while the second module 42 includes a first body member 20 and an electrode member 60. The first body member 20 is generally similar to that of FIG. 1A, except defining in its distal end 20N an annular stop 22 which bulges outwardly to a preset height. The electrode member 60 is also similar to that of FIG. 1A, except that such a member 60 may include multiple contacts 63 in the distal end 20N of the first body member 20. More particularly, such contacts 63 are preferably arranged radially along a circumference of the first body member 20. The power member 50 is also similar to that of FIG. 1A and formed of a conventional battery storing therein the electrical energy for generating electrical potential having an amplitude which may be high enough to contract or twitch various muscles of the pelvic structure of the female and/or to stimulate various nerves of the pelvic structure. The power member 50 is preferably arranged to generate at least tens (or hundreds) of such potentials so as not to run out of such electrical energy during use. Contrary to that of FIG. 1A, the power member 50 is incorporated into the first module 41 which may be provided separate from the second module 42. In addition, the power member 50 defines an annular contact 53 in its interior surface.

Although not shown in the figure, the system 10 may include a control member which may be arranged to control various operations of the system 10. A major function of the control member may be to control timing of the electric potential, i.e., a firing sequence of such electric potentials along the electrode member 60. As will be described in greater detail below, the control member may determine such timing based on various control algorithms.

In operation, the shaft of the penile structure is inserted through the lumen 20L of the first body member 20 into a preset depth, while exposing a tip of the penile structure through one end 20E of the first body member 20. In result, a major portion of the shaft of the penile structure may be enclosed or covered by the side 20S of the first body member 20 and the tip of the penile shaft is exposed through the open proximal end 20N of the first body member 20. Thereafter, the first module 41 is sled over or onto the second module 42 to a desirable depth, particularly until the annular contact 53 of the power member 50 disposed in the first module 41 touches and electrically couples with the contact 63 of the electrode member, thereby forming a circuit from the power member 50 to the electrode member 60. The stop 22 disposed in the distal end of the first body member 20 may serve as a guide to the depth. The male then inserts the shaft of his penile structure into the internal cavity of the pelvic structure of his partner through the pelvic orifice and commences reciprocating movements. Along therewith, the first body member 20 is disposed inside the internal cavity, while the electrode member 60 is brought into contact with the pelvic wall. Based on the timing control or firing sequence algorithm, the control member draws a preset amount of electrical energy from the power member 50, evokes the electric potential, and releases such potential through its contact 53. The electrode member 60 receives such potential from the power member 50 through its own contacts 63 and transmits such potential through the electrode member 60 to various portions of the pelvic structure. Because the pelvic wall is a good electric conductor and also secrets aqueous conductive fluids during sexual intercourse, the potential is delivered from the electrode member 60 to various portions of the pelvic structure, thereby causing contraction of the pelvic muscles and/or stimulation of the pelvic nerves. Other configurational and/or operational characteristics of the system 10 of FIG. 2A are similar or identical to those of the systems of FIGS. 1A to 1D.

In another exemplary embodiment of such an aspect of the invention and as described in FIG. 2B, an exemplary adaptive stimulation system 10 may similarly include a first module 41 and a second module 42, where such a first module 41 includes a first body member 20, a power member 50, and an electrode member 60, while the second module 42 includes a second body member 30. The first body member 20, power member 50, and electrode members 60 are all identical to those of FIG. 1B, so that the electromechanical generator of the power member 50 may convert the external perturbations such as the external force, momentum, and/or energy into the electrical energy and then generate the electric potential therefrom so that the electrode member 60 delivers the potential therealong to various parts of the first body member 20. The second body member 30 is disposed in the second module 42, where the second body member 30 is generally elongated, forms an annular cylinder, and defines a side 30S having an exterior surface 30E and an interior surface 30I. Bound by the interior surface 30I is a lumen 30L which also forms a shape of a cylinder. In this embodiment, the second body member 30 forms two opposing ends 30N both of which are open such that the second body member 30 may receive a preset length of a shaft of a penile structure of a male. In addition, the second body member 30 also defines a shape of a converging flange along its longitudinal axis so that a diameter of such a lumen 30L on the proximal end is larger than that of the lumen 30L on the distal side. In addition, such a second body member 30 is preferably shaped and sized to receive the shaft of the penile structure therein. Similar to the first body member 20, the second body member 30 may also be made of and/or includes elastic materials such that its axial and radial dimensions may change in response to external force. At least a part of the second body member 30 may also be made of and/or include electrically conductive materials. Alternatively, the second module 42 may include an auxiliary electrode member so as to deliver the electric potential thereacross toward the pelvic structure. When desirable, such a second member 30 may include a magnet therein or therealong or may be made of and/or include the magnetic material therein for the purpose of generating the magnetic field therearound.

Although not shown in the figure, the system 10 may include a control member which may be arranged to control various operations of the system 10. A major function of the control member may be to control timing of the electric potential, i.e., a firing sequence of such electric potentials along the electrode member 60. As will be described in greater detail below, the control member may determine such timing based on various control algorithms.

In operation, the shaft of the penile structure is inserted through the lumen 20L of the first body member 20 of the first module 41 into a preset depth, while exposing a tip of such a structure through one end 20E of the body member 20. As a result, a major portion of the shaft of the penile structure is to be enclosed or covered by the side 20S of the first body member 20 and the tip of the penile shaft is exposed through the proximal end 20N of the first body member 20. The second body member 30 is disposed over the orifice of the pelvic structure and/or inserted into the internal cavity thereof, while ensuring that the second body member 30 may be slipped off therefrom during use of the system 10. In particular, the narrow end 30N of the second body member 30 is disposed inside the internal cavity of the pelvic structure, while exposing the wider end 30N thereof over or around the orifice of such a structure. Thereafter, the male inserts the shaft of his penile structure into the internal cavity of the pelvic structure of his partner through the pelvic orifice and, accordingly, the first body member 20 is disposed inside the and/or enclosed by the second body member 30. As the male starts reciprocating movements of his penile structure inside the pelvic structure of his partner, the first body member 20 also abuts and reciprocates against the second body member 30 which may then cooperate with the first body member 20 according to a few different mechanisms. In one example, such a second body member 30 may have a rigidity which is greater than the corresponding portion of the pelvic structure, thereby applying a greater force, momentum, and/or energy onto the electromechanical generator of the power member 50. Accordingly, the power member 50 may convert such external perturbations into more electrical energy than otherwise. In another example, the second body member 30 may be shaped and/or sized to operatively couple with the power member 50 in order to facilitate movement of the movable part of the power member 50. To this end, the interior surface of the wider end 30N of the second body member 30 may form ridges and/or ripples which may abut the corresponding parts of the movable part of the power member 50. In another example, the second body member 40 may be arranged to generate the magnetic field therealong or therearound such that the electrode member 60 of the first module 41 may generate the electrical energy through electromagnetic induction. Such a second body member 30 may include a sheet- or strip-shaped permanent magnet therein or may be made of and/or include the magnetic materials therein, where one example of the latter embodiment is a composite elastic polymer in which magnetic powder is dispersed. Regardless of the exact type of the second body member 30, the control member draws a preset amount of energy from the power member 50 based on a timing control (or firing sequence) algorithm, generates such electric potential, and then releases such potential therefrom. Such potential is then transmitted through the electrode member 60 to various portions of the pelvic structure through the second body member 30. Because the pelvic wall itself is an electric conductor and also secrets aqueous conductive fluids during sexual intercourse, such potential is delivered from the electrode member 60 to various portions of the pelvic structure, thereby causing contraction of the muscles of the pelvic structure and/or stimulation of the nerves thereof. Other configurational and/or operational characteristics of the system 10 of FIG. 2B are similar or identical to those of the systems of FIGS. 1A to 1D and those of the system of FIG. 2A.

In another exemplary embodiment of such an aspect of the invention and as described in FIG. 2C, an exemplary adaptive stimulation system 10 may similarly include a first module 41 and a second module 42, where the first module 41 includes a first body member 20, and where the second module 42 includes a movable unit 54 (which corresponds to a second body member), a power member 50, and an electrode member 60. The first module 41 mainly consists of the first body member 20 which defines an elongated annular cylinder and is typically similar to that of FIG. 1A. Although not shown in the figure, the first body member 20 may include a sheet- or strip-shaped magnet or may be made of and/or include magnetic materials therein. The movable unit 54 of the second module 42 also defines an elongated annular cylinder which is shaped and sized to be movably disposed over the first body member 20. In addition and as manifest in the figure, at least a portion of the movable unit 54 is further arranged to be open along one side so as to facilitate movable disposition of the second body member 30 over the first body member 20 as well as to ensure the movable disposition of the movable unit 54 when the first body member 20 has an elastic configuration and, therefore, changes its configuration when it is fitted over the shaft of the penile structure during use. The second module 42 also includes a pair of couplers 55 which is shaped and/or sized to be releasably or fixedly disposed over the first body member 20. The couplers 55 are also arranged to be disposed on opposite ends of the movable unit 54 and to mechanically couple with such ends of the movable unit 54. In addition, the couplers 55 are made of and/or include elastic materials such that the movable unit 54 may be always positioned therebetween. As will be described in the next paragraph, the movable unit 54 of this embodiment is to function as the power member 50 which delivers the electric potential to the electrode member 60 which is also to be disposed over the movable unit 54 in a helical pattern.

In operation, the shaft of the penile structure is inserted through the lumen 20L of the first body member 20 of the first module 41 into a preset depth, while exposing a tip of such a structure through one end 20E of the body member 20. As a result, a major portion of the shaft of the penile structure is to be enclosed or covered by the side 20S of the first body member 20 and the tip of the penile shaft is exposed through the proximal end 20N of such a first body member 20. The movable unit 54 of the second body member 30 is then disposed over the first body member 20 along with the couplers 55. More specifically, one of the couplers 55 is disposed close to the base of the penile structure, while the other of such couplers 55 is positioned closer to the tip of the penile structure. Therefore, a major part of the movable unit 54 is interposed between the couplers 55 while being disposed over the shaft of the penile structure. Care should be taken, however, that the couplers 55 may not be stripped off from the first body member 20 during use. To this end, the couplers 55 may further be provided with an adjustable mechanism to manipulate a length of its circumference. Thereafter, the male inserts the shaft of his penile structure in the internal cavity of the pelvic structure through the pelvic orifice and, accordingly, the movable unit 54 abuts the muscles of the walls of the pelvic structure. As the male starts reciprocating movements of his penile structure inside the pelvic structure of his partner, such muscles may abut and resist the movement of the system 10. Because the first body member 20 is rather fixedly retained by the penile structure, such muscles may abut and retard the movement of the movable unit 54. Therefore, such a movable unit 54 tends to lag behind the inwardly moving first body member 20 during the inward penetration of the penile shaft, and then tends to stay inside the internal cavity and placed away from the penile shaft during the outward retraction of the penile structure. In short, the movable disposition of the system 10 may create a relative movement between the first and second modules 41, 42, generating the electrical energy through a few different mechanisms. In one example and as described above, the electrode member 60 may generate the electrical energy by the electromagnetic induction as the movable unit 54 slides back and forth over the magnetic field formed by the first body member 20, where the first body member 20 may include the sheet- or strip-shaped permanent magnet or may be made of and/or include various magnetic materials, where one example of the latter embodiment is a composite elastic polymer in which magnetic powder may be dispersed. In another example, the movable unit 54 may include the aforementioned electromechanical generator which may receive the external perturbations exerted by the couplers 55 and/or generate movement caused by the couplers 55. Regardless of the exact type of the movable unit 54, the control member may draw a preset amount of energy from the power member 50 based on a timing control (or firing sequence) algorithm, generate such electric potential, and then release such potential therefrom. The potential is transmitted through the electrode member 60 to various portions of such a pelvic structure. Because the pelvic wall is an electric conductor and also secrets aqueous conductive fluids during sexual intercourse, such potential is delivered from the electrode member 60 to various portions of the pelvic structure, thereby causing contraction of the muscles of the pelvic structure and/or stimulation of the nerves thereof. Further configurational and/or operational characteristics of the system 10 of FIG. 2C may be similar or identical to those of the systems of FIGS. 1A to 1D and those of the system of FIGS. 2A and 2B.

In another exemplary embodiment of such an aspect of the invention and as described in FIG. 2D, an exemplary adaptive stimulation system 10 may consist of a first module 41, whereas a second module 42 is provided as an external module which may or may not be a part of the system 10. The first module 41 includes a first body member 20 and an electrode member 60, where such a first body member 20 defines an elongated annular cylinder and is generally similar to that of FIG. 1A, while the electrode member 60 which includes multiple concentric loops of electric conductors similar to that of FIG. 1C. The second module 42 in turn includes the power member 50 which is arranged to generate the magnetic field therearound. Such a power member 50 may be battery operated for portable use, may be operated by an AC power or DC power obtained by rectifying the AC power. In addition, the system 10 may utilize an external device which may generate therearound a desirable magnetic field, where examples of such devices may also include, but not be limited to, external audiovisual devices, communication devices, computers and their peripherals, electric appliances including electric motors, electric heaters such as electric blankets, mats, and/or pads, and the like. In general, an exact shape and/or size of the power member 50 may not be material to the scope of the present invention as long as such a member 50 may generate the magnetic fields of the desired strength. It is appreciated that, in this embodiment as well as other embodiments described heretofore and hereinafter, each of such power members capable of generating the electrical energy by electromagnetic induction may include at least one movable unit and at least one stationary unit or, alternatively, may include multiple movable units. In addition, each of those power members may include at least one electrical conductor as well as at least one magnet. Therefore, the electrical conductor may be incorporated into the movable (or stationary) unit, while the magnet may be incorporated into the stationary (or movable) unit. When the power member includes multiple movable units, such an electrical conductor and magnet may then be assigned in each of such movable units. It is appreciated, however, that the direction of the magnetic field and the orientation of the electrical conductor may preferably be arranged perpendicular to each other for the most efficient generation of the electrical energy.

In operation, the shaft of the penile structure is inserted through the lumen 20L of the first body member 20 into a preset depth, while exposing a tip of the penile structure through one end 20E of the first body member 20. In result, a major portion of the shaft of the penile structure may be enclosed or covered by the side 20S of the first body member 20 and the tip of the penile shaft is exposed through the open proximal end 20N of the first body member 20. The second module 42 is then disposed close to the first module 41 within a distance in which the magnetic field generated by the power member 50 of the second module 42 may induce an electric current along the electrode member 60 of such a first module 41. Thereafter, the male inserts the shaft of his penile structure into the internal cavity of the pelvic structure of his partner through the pelvic orifice, and starts reciprocating movements. Along therewith, the first body member 20 is disposed inside the internal cavity, while the electrode member 60 is thereby brought into contact with the pelvic wall. In addition, the power member 50 creates the induced current along the electrode member 60 by the movements of the first module 41. Based upon the timing control or firing sequence algorithm, the control member releases such potential generated by the electric current to the electrode member 60 which transmits such potential to various portions of such a pelvic structure. Because the pelvic wall forms an excellent electrical conductor and also secrets aqueous conductive fluids during sexual intercourse, the electric potential is delivered from the electrode member 60 to various portions of the pelvic structure, thereby causing contraction of the pelvic muscles and/or stimulation of such nerves thereof. Further configurational and/or operational characteristics of the system 10 of FIG. 2D are similar or identical to those of the systems of FIGS. 1A to 1D and those of the systems of FIGS. 2A to 2C.

Configurational and/or operational variations and/or modifications of the above embodiments of the exemplary systems and various members thereof described in FIGS. 1A through 1D also fall within the scope of this invention.

Various members of the adaptive stimulation system of this invention may be incorporated into multiple modules of the system according to various embodiments, where Table 1 tabulates exemplary dispositions of such members in a dual-module system. It is appreciated in the Table 1 that “B” means any of the above body members (either the first body member to be disposed over the penile structure or the second body member to be disposed over the pelvic structure), “E” refers to any of the above electrode members, and “P” denotes any of the above power members.

TABLE 1
Exemplary Dispositions of Various Members in Dual-Module Systems

| Case | 1st Module | 2nd Module |
|---|---|---|
| 01 | BEP | none |
| 02 | EP | B |
| 03 | BP | E |
| 04 | BE | P |
| 05 | P | BE |
| 06 | E | BP |
| 07 | B | EP |
| 08 | none | BEP |
| 09 | BEP | BEP |
| 10 | BEP | EP |
| 11 | BEP | BP |
| 12 | BEP | BE |
| 13 | BEP | P |
| 14 | BEP | E |
| 15 | BEP | B |
| 16 | BEP | none |
| 17 | EP | BEP |
| 18 | EP | BP |
| 19 | EP | BE |
| 20 | BP | BEP |
| 21 | BP | EP |
| 22 | BP | BE |
| 23 | BE | BEP |
| 24 | BE | EP |
| 25 | BE | BP |
| 26 | P | BEP |
| 27 | E | BEP |
| 28 | B | BEP |

For example, the system according to the Case 1 consists only of the first module with such a body member, electrode member, and power member therein, as exemplified in FIGS. 1A through 1D. Conversely, the system of the Case 8 consists only of the second module which includes all of such members. In other Cases, at least one of such body, power, and electrode members is incorporated into each of the first and second modules so that the electric potential generated in one or both of the modules may be delivered to the pelvic and/or penile structures through one or both of such modules.

It is appreciated that some modules according to the above embodiments may not include any body members, as exemplified in the second modules of the Cases 2 and 3, the first modules of the Cases 5 and 6, just to name a few. In such embodiments, the power and/or electrode members may be arranged to directly contact the penile or pelvic structure, the electrode (or power) member may be disposed over (or under) the power (or electrode) member, and the like. When necessary, additional body members may be incorporated into one or both of the modules of such Cases, where such an additional body member may be relatively small or narrow, may not necessarily be disposed over or on the penile or pelvic structure, and the like. When desirable, the electrode and/or power members of such Cases may instead be arranged to releasably couple with the corresponding structure similar to the first and/or second body members of the above figures.

As manifest in the above Table 1, the system may include multiple members of the same type, in which each of such members may have the same or similar shapes and/or sizes and may perform the same or similar functions. For example, each of multiple electrode members may be included in the first and second modules, receive the electric potential from a common power member, and deliver the potential to the pelvic or penile structures, but to different portions thereof. In the alternative, each of such redundant members may be arranged to define different shapes and/or sizes and may perform different functions. For example, each of multiple electrode members may be disposed in the first and second modules, receive the electrical potential from different power members, and then deliver such potential to the pelvic and penile structures, respectively. It is to be understood that an exact number of such redundant members may not be material to the scope of the present invention as long as such a system may perform the desired function, i.e., generating the electric potential and delivering such potential to the target portion(s) of the pelvic and/or penile structures.

Regardless of the exact number of such modules, such a system may be designed such that all modules may be worn by and/or disposed only over the penile structure or, alternatively, only over the pelvic structure. In the first embodiment, such a system typically includes the body and electrode members, where the power member may be disposed on, inside or below the body member, may be positioned in other body parts of the female or male, may be disposed external to the system, where the power may be transmitted to the electrode member through wire or wirelessly, and the like. Such a system may also include the control member details of which will be provided below. In the second embodiment, the system similarly includes the body and electrode members, where the power member may be disposed over, inside or below the body member, may be positioned in other body parts of the male or female, may be disposed external to the system, where the power may be transmitted toward the electrode member through wire or wirelessly. The system may include the similar control member as well. When desirable, at least a portion of the system may be implanted into the body of the user, e.g., by implanting the electrode member or one or more spot electrodes inside the pelvic muscles, and the like;

Although the above Table 1 only tabulates various embodiments of the dual-module systems, the system may include more than two modules, where each module may include at least one of the body, power, electrode, and control members. As described in the above Table 1, some modules may not include any of the above members and may serve only for coupling purposes. In addition, at least one of such modules may be arranged to be reusable or, alternatively, at least one of such modules may be arranged to be disposable after a single use or a preset number of uses.

As described above, the adaptive stimulation system may include at least one control member for various reasons. In one example, the system may measure and monitor various variables related to the system itself, to the pelvic structure, and/or to the penile structure by such a control member. In another example, the system may manipulate various settings of its operation by itself (i.e., adaptively) or through an user intervention (i.e., manually). In another example, the system may control various timings and/or sequences of generating the electric potential and/or delivering the electric potential to the electrode member. In yet another example, the system may synchronize various dynamic features of generation and/or delivery of the electric potential with various dynamic patterns of such variables related to the system, pelvic structure, and/or penile structure. Such a control member may further be disposed in or over one of the above modules, in or on one of such body members, away from such modules, away from the system, and the like, where the control member may communicate with the rest of the system through wire or wirelessly in the latter embodiment.

Such a control member may include at least one sensor unit which may measure and monitor various dynamic patterns of the system, pelvic, and/or penile variables, which may also measure and monitor various dynamic features of such electric potentials generated by the power member and/or delivered along or to the structure through the electrode member. When desirable, the control member may also be arranged to assess other dynamic patterns and/or features from the measured dynamic patterns of the system, pelvic structure, and/or penile structure and/or from such measured dynamic features of the electric potentials.

Such sensor units may then be arranged to measure the above variables. For example, such sensor units may monitor changes in lengths thereof caused by displacement or deformation thereof, changes in surface areas or cross-sectional areas thereof, changes in volumes of such units caused thereby, changes in an angle defined between at least two preset parts of such units, changes in a curvature along at least parts of such units, and so on. It is to be understood that any of the variables defined in any directions may be measured as long as such sensor units may be properly aligned to measure such variables related to bending of such a part of the sensor unit. Alternatively, the control member may be arranged to assess the above variables from different variables measured by such sensor units. Further details of this embodiment will be provided below.

Other conventional sensors may further be incorporated into the sensor units for measuring other variables, where examples of the conventional sensors may include, but not be limited to, force sensors or transducers, velocity sensors or velocimeters, acceleration sensors or accelerometers, displacement sensors capable of measuring any of the above variables associated with displacement or deformation of at least a part of the sensor unit, contact sensors employing mechanical, electrical, magnetic, and/or chemical mechanisms, duration or timing sensors such as clocks or timers, electric current or voltage sensors (or meters) for measuring physiologic current or voltage associated with contraction and relaxation of the above muscles, and the like. Any of these sensor units may then be disposed in preset locations of the system and in preset arrangements in order to measure the above variables defined along a preset direction.

The sensor units may measure the above variables in absolute values, and the control member may provide such values to the user for monitoring purposes. In the alternative, the sensor units may measure such variables in absolute values, and the control member may then convert the values into relative values by normalizing the absolute values with respect to various preset reference values of the same or different variable, where examples of such preset reference values may include, but not be limited to, a preset value of the same (or different) variable determined by a manufacturer or user, a preset value of the same (or different) variable averaged over a preset time interval and/or a preset area, a peak or maximum (or minimum) value of the same (or different) variable, a value of the same (or different) variable obtained in a proceeding measurement, and so on. Such values may further be obtained in an analog format or a digital format. In another alternative, the sensor units may measure the variables in one or more of the above relative values, where the control member may provide such relative values to the user. In addition and as described hereinabove, the sensor units and/or control member may measure and provide such absolute and/or relative values of the above variables to the user without performing the baseline adjustment or after adjusting such a baseline.

The control member may include any number of such sensor units which may be disposed in almost any arrangements and which may measure the same or different variables which are defined in the same or different portions of such structures. In one example, the control member may have at least two sensor units which may monitor the same variable and may be disposed in different parts of the body member. In another example, the control member may have at least two sensor units which may be disposed in a preset part of the body member and measure different variables. In yet another example, the control member may include at least two sensor units which may instead be disposed in different parts of the body member and measure different variables. When desirable, the sensor units may be disposed one over the other or side by side. In addition, at least one of such sensor units may also be disposed in the second unit and/or stop defined along the body member.

Such sensor units may be defined in various parts of the system. When the system includes multiple sensor units, at least two of such units may be identical, similar or different, at least two of such units may be disposed close to each other, away from each other, or one above the other.

The system may play sound and/or display an image of the variable and/or value thereof with an internal audio output unit, an internal visual output unit, and/or an internal audiovisual output unit. In case of storing the signals, the system may also include an internal audio input unit, an internal visual input unit, and/or an internal audiovisual input unit. Such sound and/or image may then be generated in response to the measured or assessed value of the variable which may be effected or initiated by the user or may be generated to effect such a variable with such a value by the user.

Such sensor units may be disposed in preset strategic locations along the body member so as to measure the variables defined in, on or around a clitoris of the entry of the pelvic structure, G spot on the pelvic wall, other portions of the wall, and the like.

Each of the above variables may further define dynamic patterns which may be one or both of temporal patterns and spatial patterns, where examples of the temporal pattern may include, but not be limited to, an instantaneous value of the variable, a time-varying (or time-dependent) value thereof, a time-averaged value thereof, an average thereof weighted by a preset weighting functions, a peak value thereof, a time derivative thereof in the first, second or higher order, an integration thereof over time, and the like, while examples of the spatial pattern may include, but not be limited to, a localized value of the variable, a spatial distribution thereof, an area-averaged value thereof, its global or local peak in a preset domain (e.g., a preset area or volume), a spatial derivative thereof in the first, second or higher order, a spatial derivative thereof along one or more directions, an integration over a preset length, area or volume, and the like.

The temporal patterns of the above variables may also include a duration of such a variable, its frequency, its temporal sequence, and the like, and the spatial patterns may include an amplitude of the variable, its direction, and the like. The dynamic pattern may further include a frequency of such a variable, its temporal rate of change (or temporal differentiation), its displacement (or its integral over time) caused thereby, and a compound value obtained by at least one of mathematical manipulation of at least one thereof. In addition, the dynamic pattern may include a duty cycle of any of the variables such as, e.g., periods in which such variables may change their amplitudes and/or direction, areas in which such variables may be defined, and the like.

In addition, such variables may also include normal force applied to at least a part of the body member, bending force applied to such a member, axial force pulling or pushing the member into or out of the internal cavity, torque applied around the member, velocity of the member, acceleration of such a member, displacement of the member, contact between the member and a corresponding portion of the pelvic or penile structure, a dimension of the portion of the structure, contraction and relaxation of the portion, a duration of at least one of such variables, a frequency of at least one of such variables, and the like. In the alternative, such variables may include normal force applied onto at least a portion of the pelvic or penile structure, bending force applied to the portion, axial force resisting movement of the first unit into (or out of) the cavity of the structure, torque applied around such a portion, velocity of the portion, acceleration of the portion, displacement of the portion, contact between such a portion and such a member, contraction and relaxation of such a portion, a duration of at least one of such variables, a frequency of at least one of the variables, and the like. Such force may also be a torque effected about an axis of rotation or pivoting of at least a part of the sensor unit or at least a portion of the structure. It is appreciated that such sensor units including the force sensors may be made of and/or include elastic and/or deformable materials so as to deform in response to such force. In the alternative, such sensor units may maintain its configuration during measuring the above variables by employing rigid sensors such as, e.g., piezoelectric sensors.

Each of such temporal and/or spatial patterns (i.e., the dynamic pattern) of the variable may be directly measured by the sensor unit or, alternatively, may instead be assessed from other measured patterns, where such assessment may then be performed directly by the sensor unit or by the control member. For example, the sensor unit may be the force transducer (or pressure sensor) capable of measuring various forces or pressures applied thereto or applied onto at least a portion of the pelvic structure, while the control member may assess therefrom acceleration of the sensor unit and/or the portion of the pelvic structure, velocity of the part of the sensor unit and/or of the portion of the pelvic or penile structure, displacement of the body member and/or portion, mass of such a member and/or portion, momentum of such a member and/or portion, mechanical energy associated with the member and/or portion, duration of the force (or pressure), frequency of the force (or pressure), and the like. In another example, the sensor unit may be the displacement sensor capable of measuring extents of the deformation or displacement of at least a part of the sensor unit and/or at least a portion of such a pelvic or penile structure, and the control member may assess therefrom acceleration of such a part and/or portion, velocity of the part and/or portion, mass of the part and/or portion, force (or pressure) applied to the part and/or portion, momentum associated with such a part and/or portion, mechanical energy associated with the a part and/or portion, duration of the movement of the part and/or portion effecting such displacement or deformation, frequency of such movement, and the like.

Still referring to the same variation or modification, the sensor unit may be the velocity sensor capable of measuring the velocity of at least a part of the sensor unit and/or at least a portion of the pelvic or penile structure, while the control member may assess therefrom acceleration of such a part and/or portion, displacement of such a part and/or portion, mass of such a part and/or portion, force or pressure applied onto of the part and/or portion, momentum associated with the part and/or portion, mechanical energy accompanying such a part and/or portion, duration of movement of the part and/or portion which may cause such a velocity, frequency of such movement of the part and/or portion, and the like. It is appreciated that the sensor unit may measure or assess the distance to the pelvic and/or penile structures, velocity of the part and/or portion, and the like, by measuring the distance to such a structure. In another example, the sensor unit may be the acceleration sensor capable of measuring such acceleration of at least a part of the sensor unit and/or at least a portion of the pelvic or penile structure, and the control member may then assess therefrom velocity of such a part and/or portion, displacement of the part and/or portion, mass of the part and/or portion, force or pressure applied to the part and/or portion, momentum related to the part and/or portion, mechanical energy related to the part and/or portion, duration of movement of such a part and/or portion effecting such acceleration, frequency of the movement, and the like. In another example, the sensor unit may be made from any conventional sensor capable of monitoring electrical, mechanical, magnetic, and/or chemical contact between at least a part of the body member and at least a portion of the structure, while the control member may assess therefrom a duration of the contact, a frequency of such contact, and the like. When desirable, the sensor unit may include any conventional optical sensors to detect such contact. It is to be understood that such sensor units for detecting such contacts may be arranged to operate based upon a preset threshold. Accordingly, the sensor unit for detecting mechanical contact may be arranged to detect such contact only when the sensor unit may be disposed within a preset distance from the pelvic or penile structure, only when the structure exerts force with an amplitude exceeding the threshold, and the like. Similar provisions may also be applied to other sensor units for electrically, magnetically, optically, and/or chemically detecting such contact.

In addition to the above variables, the sensor units may also be arranged to measure a shape and/or size of at least a portion of the pelvic or penile structure. For example, the sensor unit may be arranged to measure the diameter or radius of a preset portion of such a structure, the length or depth into a preset portion thereof, and the like. It is preferred, however, that measurements be performed after the baseline adjustment of the sensor units in order to measure more accurate dimension of the portion of the structure, where details of such baseline adjustment have been provided in the above co-pending Applications.

The sensor units may also measure electric voltages and/or currents representing contraction and/or relaxation capabilities of the pelvic or penile muscles. Any conventional voltage and/or current meters may be employed for such purposes. Alternatively, such sensor units may also measure the voltages and/or currents generated by the system and delivered to such pelvic or penile muscles. In this embodiment, the sensor units may also measure other variables of the pelvic oe penile structure which are evoked in response to such voltages and/or currents.

Various clocks and/or timers may also be employed as the sensor units and measure various timings and/or durations associated with any of the above variables. Accordingly, such sensor units for measuring the timings, durations, and/or frequencies may be used in conjunction with other sensor units in order to determine such temporal patterns of such variables.

It is to be understood that such sensor units may measure some of the above variables such as, e.g., displacement or deformation of at least a part of the sensor unit and/or at least a portion of the pelvic or penile structure, velocity of such a part and/or portion, and acceleration of such a part and/or portion, in various arrangements. In one example, the sensor units may deform or move while measuring the variables. In another example, the sensor units may maintain their shapes and/or sizes while measuring such variables but such sensor units may be incorporated into a preset part of the body member which may deform or move during such measurements. In another example, the sensor units may maintain their shapes and/or sizes during such measurements.

The control member may also include at least one storage unit which may store various signals and/or control algorithms for generating and/or delivering the electric potential, where further details of such algorithms will be provided below. In addition, the storage unit may also be arranged to store an unused portion of the electrical energy, undischarged electrical energy, and the like.

Although not included in the figures, the system may include other members and/or unit which may output to the user values of various physiologic variables of the pelvic structure monitored by the sensor unit. For example, the system may include at least one audio and/or visual output unit so that the values of the monitored pelvic or penile variables may be provided to the user audibly as sounds and/or visually as images. Details of such output units are provided in the co-pending Applications.

The adaptive stimulation system of this invention may be provided as an unitary article. In such an embodiment, the body member of such a system may incorporate therein various other members or units of the system. Alternatively, the system may include the power and/or control members which may be detached from the body member but operatively couple with the electrode member disposed on the body member wirelessly or through wire. When desirable, such a system may be arranged to utilize external audiovisual devices for playing various sounds and/or displaying various images. The system may be operatively coupled to such external devices through wire or wirelessly. In addition, such a system may be arranged to only include the control member which may then imports various signals related to various pelvic or penile variables. The adaptive stimulation system of this invention may be construct to be waterproof. For example, various members may be covered by a waterproof layer or may be disposed inside the body member. In addition, the system may be arranged to run by a rechargeable battery which may be recharged by electromagnetic induction from outside.

Unless otherwise specified, various features of one embodiment of one aspect of the present invention may apply interchangeably to other embodiments of the same aspect of this invention and/or embodiments of one or more of other aspects of the present invention. Accordingly, various power and/or electrode members of FIGS. 1A to 2D may be replaced interchangeably without departing from various scopes of the present invention. Similarly, any of the sensor units disclosed hereinabove may be incorporated into any of the adaptive stimulation systems as well.

The adaptive stimulation system of the present invention may also be used in conjunction with various audiovisual aids. For example, the user may play a source of audio signals which generates various audible sounds to which the user may synchronize her sexual activity, based upon which the user may engage in the activity, and the like. The control member may then be arranged to receive the audio signals, to compare such audio signals with the sensing signals generated by the sensor units, and to provide the user with the feedback audiovisual signals. In another example, the user may turn on a source of visual signals generating various visual signals to which the user may synchronize the activity, based upon which the user may perform the activity, and so on. The control member may be arranged to receive the visual signals, to compare the visual signals with the sensing signals which are generated by the sensor units, and to provide the user with such feedback audiovisual signals. In yet another example, the user may play another visual signals generating various images to which the user may synchronize the sexual activity, based upon which the user may perform the activity, and the like. The control member may be arranged to receive the visual signals, to compare such signals with the sensing signals generated by the sensor units, and to provide the user with such feedback audiovisual signals.

As described above, the control member of the adaptive stimulation system may operate under various control modes. In one example, the control member may operate based on various command signals provided thereto manually by the male or female. The user may then supply such signals with his or her finger, hand, foot, leg, arm, shoulder, thigh, leg or other body portions. When desirable, the control member may be arranged to change its control setting by receiving the control signals from the pelvic and/or penile structures. In another example, the control member may also manipulate various operations of the system adaptively, i.e., according to various system, pelvic, and/or penile variables which are measured by the above sensor units. In another example, the control member may control such operations based upon external command signals supplied thereto through wire or wirelessly from external devices such as, e.g., external audiovisual devices, communication devices, internet, and the like. In general, such a control member may be arranged to control the timings, intervals, and sequences of generation and/or delivery of the electric potentials. Such a control member may also control temporal and/or spatial patterns of such electric potentials, various dynamic features of such electric potentials, and the like. The control member may be disposed in the first and/or second body members, in the first and/or second modules, and the like, where multiple control members may then be arranged to perform the similar or different functions when incorporated into multiple modules.

Another important function of the control member is to synchronize the generation and delivery of the electric potential with various dynamic patterns of the system, pelvic, and/or penile variables. In general, details of such synchronization depend on various design factors such as, e.g., types of the power member, mechanisms of generating and delivering the potential, configuration of the electrode members, and the like.

In one exemplary embodiment, such a control member may synchronize the generation and/or delivery of the electric potential with various dynamic patterns of the power member which generates the potential by movement of at least a part thereof, e.g., as exemplified in FIGS. 1B to 1D and FIGS. 2B to 2D. For example, the control member may measure one or more variables related to a displacement or distance of movement of at least a part of the power member or other members of the system and synchronize the generation and/or delivery of the electric potential therewith, where examples of the displacement-related variables may include, but not be limited to, the displacement of the part, a speed or velocity of such a part, an acceleration thereof, a duration of such a variable, a frequency thereof, and other dynamic patterns related thereto. In another example, the control member may perform the synchronization based on absolute values of such variables, regardless of their directions. In another example, the control member may perform such synchronization while accounting for the directions of the variables, where examples of such directions may include, but not be limited to, a direction inward (or into) the internal cavity of the pelvic structure, a direction outward (or out of) the internal cavity, a direction vertical or horizontal to such a structure, an angular direction about an axis of the cavity, and the like. In a related embodiment, the control member may synchronize the generation and/or delivery of such electric potential with various dynamic patterns of stroke of the system. For example, such a control member may be arranged to generate and/or to deliver a preset number of potentials per each stroke as measured by the above sensor units. In another example, the control member may perform such synchronization in a preset landmark defining a specific point of the stroke, e.g., when the tip of the penile structure attains a maximum penetration depth per each stroke and/or per a preset number of strokes, when the shaft of the penile structure approaches the clitoris and/or G-spot, and the like. Thereafter, the control member may generate the electric potential and/or deliver such potential to the electrode member based on a value of such a variable, e.g., when such a value may exceed a preset threshold, when the value may fall within a preset range, when the value may not change more than a preset value in a preset interval, when the value may change more than the preset value in such an interval, and the like.

In another exemplary embodiment, the control member may synchronize the generation and/or delivery of the electric potential with various dynamic patterns of the power member which generates the potential by mechanical contact and/or electrical contact between at least a part of the system and at least a portion of the pelvic and/or penile structure, e.g., as exemplified in FIGS. 1B, 1C, and 2B. For example, the control member may measure one or more variables related to such a contact between at least a part of the system and at least a portion of the pelvic or penile structure and/or between at least two parts of the system, and synchronize the generation and/or delivery of the electric potential therewith, where examples of the contact-related variables may include, but not be limited to, a force, momentum or energy associated with such a contact, a location of the contact, a duration thereof, an interval between multiple contacts, a change in locations of such contacts, and the like. As described above, such contacts may be measured mechanically, electrically, optically, chemically, magnetically, thermally, and/or other means conventionally used in the relevant art. Such variables may further be assessed from other variables of the system, pelvic structure, and/or penile structure when it may not be feasible to directly measure the contact-related variables, where examples of such other variables may include, but not be limited to, a location of such a part(s) of the system, a velocity or acceleration thereof, and the like. Thereafter, the control member may generate the electric potential and/or deliver such potential to the electrode member based on a value of such a variable, e.g., when such a value may exceed a preset threshold, when the value may fall within a preset range, when the value may not change more than a preset value in a preset interval, when the value may change more than such a preset value in such an interval, and the like.

In another exemplary embodiment, the control member may synchronize the generation and/or delivery of the electric potential with various dynamic patterns related to disposition of at least a part of such a system. For example, such a control member may measure one or more variables related to the disposition of such a part of the system, and synchronize the generation and/or delivery of such electric potential therewith, where examples of such disposition-related variables may include, but not be limited to, a location of such a part with respect to a preset reference or landmark, a distance to or from the reference, an elevation above and/or below such a reference, a direction to the reference, and the like. The above reference may be selected from a variety of sources, where examples of the references may include, but not be limited to, one or more landmarks of the pelvic or penile structure, the power member which may be disposed in any of the above body members and modules or which may be external to such a member, module, and/or system, the sensor unit, other external references defined by the above external devices, and the like. Such variables may also be assessed from other variables of the system, pelvic structure, and/or penile structure when it is not feasible to measure the disposition-related variables directly, where examples of such other variables may include, but not be limited to, a location of such a part(s) of the system, a velocity or acceleration thereof, and the like. Thereafter, the control member may generate the electric potential and/or deliver such potential to the electrode member based on a value of such a variable, e.g., when such a value may exceed a preset threshold, when the value may fall within a preset range, when the value may not change more than a preset value in a preset interval, when the value may change more than such a preset value in such an interval, and the like. When desirable, the control member may synchronize such generation and/or delivery of the electric potential with various dynamic patterns related to posture of the pelvic and/or penile structures and/or related to orientations and/or movements of various portions thereof, where details of such synchronization algorithm is generally similar to those described above.

In addition, the control member may also synchronize various dynamic features of the electric potential with one or more of the aforementioned variables of the system, the pelvic structure, and/or the penile structure. In general, the control member may vary various temporal and/or spatial features of such potentials, where details of such dynamic features have been described hereinabove.

The adaptive stimulation system may also be used by being disposed over various pelvic and penile relaxing systems, details of which have been provided in the above co-pending Applications. Accordingly, such a system may be tailored to be used with conventional dildos or other novel pelvic relaxing systems of the co-pending Applications. In addition, such a system may be worn on or over a finger, a fist, and/or other objects insertable into the internal cavity of the pelvic structure. It is to be understood that, when the system is to be worn by the female, such a system may be constructed similar to a conventional female condom which, however, includes the novel members of the present invention such as the power, electrode, and/or control members as described herein.

When desirable, the adaptive stimulation system may also include auxiliary electrode members which are to be disposed in various body portions of the male and/or female excluding the pelvic and penile structures. Therefore, such an auxiliary electrode member may be disposed in other sensitive portions of the body such as, e.g., a nipple, an arm pit, a back, and other portions of the body.

It is appreciated that the Disclosure Documents which have been referred to in the section of “Cross-Reference” and bear the Ser. Nos. 611,016, 611,023, 611,027, 711,331, 611,334, and the like, have been referred to herein as the “co-pending applications.”

It is to be understood that, while various aspects and embodiments of the present invention have been described in conjunction with the detailed description thereof, the foregoing description is intended to illustrate and not to limit the scope of the invention, which is defined by the scope of the appended claims. Other embodiments, aspects, advantages, and modifications are within the scope of the following claims.

What is claimed is:

1. A stimulation system capable of providing electric potential in order to at least one of stimulate nerves and contract muscles of a pelvic structure of a female including an entry and a wall, wherein said entry defines therethrough an orifice and wherein said wall is configured to include said muscles and nerves and to form an internal cavity extending inwardly and bound by said muscles, said system comprising:
at least one first body member which is configured to receive thereinto at least a portion of a penile structure of a male;
at least one power member which is configured to provide power for said potential;
at least one electrode member which is configured to be disposed on said first body member and to receive said potential from said power member; and
at least one control member which is configured to operatively couple with said electrode and power members and to also include at least one sensor unit and at least one control unit, wherein said sensor unit is configured to measure at least one variable and wherein said control unit is configured to deliver said potential from said power member to at least one of said structures by said electrode member when a value of said variable exceeds beyond a preset value, thereby causing at least one of contraction of said muscles of said pelvic structure and stimulation of said nerves thereof.

2. The system of claim 1, wherein said variable includes at least one of a displacement of said at least one of said body members, a velocity thereof, an acceleration thereof, a direction of at least one of said displacement, velocity, and acceleration, a duration thereof, and a frequency thereof.

3. The system of claim 1, wherein said variable includes at least one of force of its contact with at least one of said structures, a position of said contact, a duration thereof, and a frequency thereof.

4. The system of claim 1, wherein said variable includes at least one of a insertion depth thereof along said internal cavity, a distance to a preset reference point therefrom, its orientation with respect to said reference point, a direction to said reference point therefrom, and an elevation with respect to said reference point.

5. The system of claim 1, wherein said power member is configured to store therein energy for generating a plurality of said potentials.

6. The system of claim 1, wherein said power member is configured to have therein at least one movable part capable of moving in response to movement of said first body member and to convert said movement into said potential.

7. The system of claim 1, wherein said power member which is configured to contact said pelvic structure, to receive contact force therefrom, and to convert said force into said potential.

8. The system of claim 1, wherein said power member is configured to include at least one of an electric conductor and a magnet each capable of moving in response to movement of said first body member, to induce electromagnetic induction when moving through at least one of external magnetic and electric fields, and to generate said potential therethrough when moving along with said first body member through said at least one of said fields.

9. The system of claim 8, wherein said external fields are static fields.

10. The system of claim 1, wherein said control member is configured to deliver said potential in at least a substantial synchronization with at least one dynamic pattern of said variable.

11. The system of claim 1, wherein said first body member is configured to be insulative such that at least a substantial portion of said penile structure is insulated from said potential.

12. The system of claim 1, wherein said first body member is configured to be conductive so that said potential is delivered to at least a portion of said penile structure.

13. The system of claim 1, wherein said first body member defines an exterior surface as well as an interior surface and wherein said electrode member is configured to be exposed through both of said surfaces of said first body member so that said potential is delivered to at least a portion of said penile structure.

14. The system of claim 1, wherein said first body member is configured to define a shape of an elongated tubing having an annular side and a pair of opposing ends one of which is open while the other of which is closed, to enclose a tip of a penile structure of said male by said closed end, and to enclose at least a portion of a shaft of said penile structure with said side.

15. The system of claim 1, wherein said first body member is configured to define a shape of an elongated tubing having an annular side and a pair of opposing open ends and to wrap around at least a portion of a shaft of a penile structure of said male while leaving a tip of said penile structure to be exposed through one of said open ends.

16. The system of claim 1, wherein said first body member is configured to define a shape of a sheet having a pair of opposing edges capable of releasably coupling onto each other and to wrap around at least a portion of a shaft of a penile structure of said male when said edges are coupled to each other.

17. A stimulation system capable of providing electric potential in order to at least one of stimulate nerves and contract muscles of a pelvic structure of a female including an entry and a wall, wherein said entry defines therethrough an orifice and wherein said wall is configured to include said muscles and nerves and to form an internal cavity extending inwardly and bound by said muscles, said system comprising:
at least one first body member which is configured to receive thereinto at least a portion of a penile structure of a male;
at least one power member which is configured to have at least one movable part capable of moving in response to movement of said first body members and to convert said movement into said potential;
at least one electrode member which is configured to be disposed on said first body member and to receive said potential from said power member; and
at least one control member which is configured to be operatively coupled to said power and electrode members and to deliver said potential generated from said movement to at least one of said pelvic and penile structures by said electrode member, thereby causing at least one of contraction of said muscles of said pelvic structure and stimulation of said nerves thereof.

18. The system of claim 17, wherein said power member is configured to store said potential until said potential reaches a preset value and to deliver said potential to said pelvic structure only when said potential exceeds said preset value.

19. A method of providing electric potential only on preset occasions for at least one of stimulating nerves of a pelvic structure and stimulating muscles of said pelvic structure of a female, wherein said pelvic structure includes an entry and a wall, wherein said entry is configured to define therethrough an orifice, and wherein said wall is configured to include said muscles and to define an internal cavity extending inwardly and bound by said muscles, said method comprising the steps of:
disposing at least one conductive electrode member on at least one first body member;
placing said first body member on at least a portion of a penile structure of a male;
engaging said first body member with said pelvic structure;
measuring at least one variable related to at least one of said structures; and
providing said potential to said pelvic structure when a value of said variable exceeds a preset value, thereby causing at least one of contraction of said muscles and stimulation of said nerves.

20. The method of claim 20 further comprising the step of:
performing said providing said potential in synchronization with at least one dynamic pattern of said variable.
